(19) United States
(12) Patent Application Publication
(10) Pub. No.: US 2022/0015698 A1
(43) Pub. Date: Jan. 20, 2022

(54) METHOD OF IDENTIFYING TUMOR DRUG RESISTANCE DURING TREATMENT

(71) Applicant: Lan Jiang, DALLAS, TX (US)

(72) Inventor: Lan Jiang, DALLAS, TX (US)

(21) Appl. No.: 17/488,396

(22) Filed: Sep. 29, 2021

Related U.S. Application Data
(63) Continuation-in-part of application No. 16/708,857, filed on Dec. 10, 2019.
Application No. 16/708,857 is a continuation-in-part of application No. 15/275,897, filed on Sep. 26, 2016, now abandoned.
(60) Provisional application No. 62/233,682, filed on Sep. 28, 2015.

(51) Int. Cl. A61B 5/00; A61B 5/055; A61B 5/026; G16H 30/40; G16H 20/10; G16H 20/40; G16H 50/70

(52) U.S. Cl. CPC A61B 5/4848; A61B 5/055; A61B 5/0036; A61B 5/0263; G16H 50/70; G16H 30/40; G16H 20/10; G16H 20/40; A61B 5/743

(57) ABSTRACT

A method of identifying tumor treatment resistance is provided. In some embodiments, the method may include: detecting tumor oxygenated blood perfusion region inside tumor by having a patient breathe air to acquire Mill baseline data; inhalation of hyperoxia gas to generate higher than baseline HbO₂ blood circulating in body to acquire MRI enhanced data; the region-of-interest (ROI), which in this case is a tumor volume (V), and which may be performed by volume contour tracing/region-of-interest (ROI) analysis and 3D tumor volumetry methods; calculating voxel's enhanced signal intensity (ΔSI); calculating tumor oxygenated perfusion percentage (OPP %); selecting different threshold and calculating maps such as a reconstruction OPP % pseudo color map; calculating tumor volume change ratio (Vt %); overlaying reconstruction OPP % pseudo color map to original images for visualizing tumor response data; drawing or plotting the OPP % and Vt % on a cancer treatment response information diagram, and identifying the type of drug resistance, classifying the drug resistance being caused by poor drug distribution factor or cells-specific factor based on pooled collected data.

### CROSS REFERENCE TO RELATED APPLICATIONS

This application is a continuation-in-part of U.S. Non-Provisional application Ser. No. 16/708,857, filed on Dec. 10, 2019, entitled “METHOD FOR PRECISION CANCER TREATMENT BY IDENTIFYING DRUG RESISTANCE”, which is a continuation-in-part of U.S. Non-Provisional application Ser. No. 15/275,897, filed on Sep. 26, 2016, entitled “CANCER THERAPEUTIC WINDOW EVALUATION METHOD”, which claims the benefit of U.S. Provisional Application No. 62/233,682, filed on Sep. 28, 2015, entitled “CANCER TREATMENT EVALUATION METHOD”, the entire disclosures of which are incorporated by reference herein.

### FIELD OF THE INVENTION

This patent specification relates to the field of identification methods of tumor drug resistance during treatment. More specifically, a non-invasive method for early identifying the type of solid cancer drug resistance. This patent specification also relates to computer implemented method of identifying drug resistance in real time during cancer treatment process.

### BACKGROUND

Although there are multiple therapeutic modalities (Chemotherapy, Radiotherapy, Immunotherapy, Molecular Targeted Therapy, etc.) available for cancer treatment in the clinical practice, clinicians still face the challenge of selecting the right therapeutic approach for right patient and balancing relative benefit with risk to achieve the most successful outcome. However, the clinical treatment effect is greatly affected by drug resistance of individual tumor. Individual differences in drug resistance greatly increase the difficulty of treating cancer, which is highly associated with the cancer angiogenic system and characteristic of cancer cells. The microcirculation of tumor plays a role of key importance during tumor growth, metastasis, and treatment. Tumor angiogenic system usually demonstrated inefficient in blood flow, oxygen and nutrition delivery comparing with normal tissue, which may directly cause the inefficient effectiveness in systemic therapies and radiotherapy. This characteristic of barrier tumor therapy is called treatment resistance or drug resistance. For example, the poor microcirculatory perfusion regions of tumor can cause suboptimal the ability of drug/agent distribution in systemic therapy inside tumor which may lead to below drug minimum effective concentration inside tumor cells and treatment failure in blood-borne therapies (Chemotherapy, Targeted therapy, Immunotherapy, Gene therapy, and Photodynamic therapy, etc.). Meanwhile, the tumor microcirculatory perfusion can be longitudinally changed with change of tumor volume (such as, shrinking or swelling) during treatment course, which also may cause huge variation in clinical treatment resistance and following outcomes. Because of the high heterogeneity of microcirculatory perfusion and oxygenation level both inter- and intra-tumor, it is one of main reasons that the same stage patients with the same treatment strategy can vary in outcome among patients. In addition, tumor cells may have vastly different responses to drugs/agents in systemic therapies due to difference in cancer cells with different DNA mutations and genomic information, which may cause treatment failure to response to the same drug/agent. Numerous studies have shown that the therapeutic resistance can be divided into two broad categories: cells-specific factors and pharmacological/physiological factors. Different types of resistance may require different treatment strategies to overcome the resistance disorders in the clinical practice. So far, the clinical identification of the treatment resistance must be close to end or after completing the relevant course of treatment. And it is hard to identify the type of tumor drug resistance during treatment. This means that a large number of patients may have to be at risk that the treatment they receive may not be effective.

Timely monitoring and identifying the type of drug resistance of tumor will greatly benefit to developing or adjusting the optimal treatment plan during treatment course. It is of significance in reducing ineffective treatment or even ineffective over-treatment in the clinical setting. Unfortunately, the research shown that majority of human tumors are inefficient microcirculation which means the necessity and importance of identifying drug resistance in systemic therapies. For example, clinical statistics report that almost 70% of breast cancer patients may not respond to chemotherapy drug. Some of these may be caused by cell-specific factors, some of which may be due to low drug distribution and low concentration causing by tumor poor microcirculation. However, although clinicians know the fact that only 30% of patients may have completely or partially clinical response, all cancer patients may still have to undergo the same chemotherapy regimen as a routine standard procedure without any information of tumor drug resistance. It will directly lead to a wide range of ineffective treatment, even ineffective overtreatment, which can damage the patient's health and waste treatment window time. Currently, there are no techniques or methods available to accurately identify the type of drug resistance during tumor treatment process.

Therefore, a need exists for novel methods of precision medicine which are able to provide the individualization of each patient's treatment for improving efficiency, which offers the ability of matching the right treatments to the right patients at the right time point to improve patient outcomes and quality of life. There also exists a need for novel method for identifying drug resistance as routine for reducing both exposure to ineffective therapies and the cost of cancer care. There is a further need for novel cancer technique platform which are able to visually aid in identifying, tracking, evaluating, and optimizing cancer therapy for customized evidence-based cancer treatment. There exists a need for novel technologic method that can help to timely identify the type of drug resistance during the course of treatment or even before treatment. There exists a need for novel method that can provide treatment information of different therapeutic modalities on one universal platform for sharing with clinicians who are different therapeutic backgrounds, comprehensively analyzing treatment response, and searching the best therapeutic strategy. Finally, there exists a need for novel platform that provide the ability of real-time monitoring therapeutic previous response and future possible response information in adjusting and optimizing of current treatment plan during treatment course for achieving precision cancer treatment in clinical setting.

### BRIEF SUMMARY OF THE INVENTION

The invention is to develop non-invasive method for evaluating tumor drug distribution, design a special information diagram reflecting the tumor treatment response, establish a set of clinical identification standards for classifying the type of drug resistance. The present invention can include four technical aspects. In some embodiments, the invention may include:

1. Quantitatively Evaluating Tumor Oxygen/Drug Distribution Characteristics

Pharmacokinetics refers to what happens to a medication from entrance into the body until the exit of all traces. Think of pharmacokinetics as a drug's journey through the body, during which it passes through four different processes: absorption, distribution, metabolism, and excretion. Each of these processes is influenced by the route of administration and the functioning of body organs. The drug distribution may directly be influenced by the microcirculation of individual tumor and is important factor to affect the therapeutic effect. Poor tumor microcirculation can directly lead to decreased tumor blood perfusion, barrier of drug distribution during delivery, and decreasing drug accumulation minimum effective concentration in tumor cells. In other words, the ability of drug distribution inside tumor region is directly correlated to drug effectiveness during systemic treatment. However, there is technically difficult to directly measure the plasma concentration of drug inside tumor region in the clinical routine. When fresh blood (high oxyhemoglobin blood) is flowing through the tumor area, it is similar to drugs delivery process inside tumor region via local microcirculation. The more areas of the tumor area where fresh blood flows through, the more oxygenated blood distribution inside tumor, the better tumor microcirculation, the better ability of the drugs delivery and drug distribution in the tumor area. The first part of present invention is to describe a novel method to evaluate the ability of tumor drug distribution via assessing the ability of fresh blood through the tumor area using an endogenous contrast enhancement MM technique.

Using endogenous contrast agent dHbO₂ and applying special imaging protocol of T2 weighted Mill technique and special data processing algorithm, the capability of fresh blood (high oxyhemoglobin, HbO₂) flowing through tumor area can be detected. By analyzing the percentage of fresh oxygenated blood flowing through tumor area, it can be used to evaluate the ability of the drug distribution during treatment course. Medical research demonstrated that tumor microcirculation, as prognostic information, is associated with the effects of systemic therapy and radiotherapy, which indicates the consistency of drug distribution in pharmacokinetic/physiological sense. In the present invention, the ability of tumor drug delivery and distribution can be quantitatively analyzed by measuring the percentage of tumor fresh oxygenated perfusion region via non-invasive Mill technique. The higher the percentage of oxygenated perfusion region in tumor area, the better the microcirculation of the tumor, the better drug delivery and distribution in tumor area. The advantage of using endogenous contrast agent via new T2-weighted Mill imaging protocol and data processing algorithm can be used to monitor and evaluate the drug distribution of tumors during the treatment process, and it can solve the current clinical measurement problem of tumor microvascular permeability changing.

2. Design a Specific Infographic (“the Diagram”) and Establish a Unified Standard for Visualizing and Assessing Tumor Therapeutic Response

Two parameters of the previous response (tumor volume change) and future possible response (tumor drug distribution) are very important therapeutic information for evaluating tumor response to treatment. Changes in tumor volume are used to assess treatment outcomes objectively. However, tumor volumes in response to effective treatment are significantly delayed by a few days or weeks. The tumor volume information only reflects a result of previous treatment when measuring tumor during treatment course. At the same time, changes in tumor volume will inevitably lead to changes in tumor microcirculation and the ability of drug distribution, which means that previous treatment response may not predict future treatment response which may have different outcomes. The clinicians are eager to know the both information to analyze the previous treatment response of the tumor and the future possible therapeutic response in order to optimize treatment plan in time. The tumor volume change and ability of drug distribution can be integrated a tumor response point in a two-dimensional coordinate system that represents previous treatment outcomes and possible future treatment outcomes. Visualization of multiple tumor response points (two-dimensional tumor response information) can be used to monitor treatment progress and identify different treatment resistances, thereby optimizing treatment strategies and reducing ineffective treatment.

In order to accurately understand treatment response information of individual tumor, all treatment response information may be aggregated in one diagram of infographic with uniform criteria. In the present invention, a novel specific cancer treatment response information diagram is designed for displaying all treatment response information points on one platform. By visualizing all treatment responding points in one diagram, clinicians even patients can easily understand the treatment progress and prognosis. It can greatly help clinicians master the progress of treatment, optimize treatment plans in time, and reduce ineffective treatment. In order to distinguishing different therapy modalities, the results of systemic therapy and radiotherapy can be displayed in two symmetric coordinate graphs respectively, which may be used to help clinicians verify the effects of different treatment strategy. The relative value of the parameters as a uniform standard may be suitable for analyzing all solid tumor cases on a single diagram.

3. Establish a Classification Method to Identify the Type of Drug Resistance

The accurate identification of drug resistance and its type has important clinical significance and is also the goal of the present invention. Clinical studies have shown that drug resistance in systemic therapy can be divided into two broad categories: cell-specific factors and pharmacological/physiological factors. The different type of drug resistance may have to take different therapeutic strategies to overcome their barriers. Timely identification of the type of resistance is extremely important for Precision Medicine in Cancer Treatment. Clinical research found that majority of human tumors represent inefficient microcirculation. Drug resistance caused by poor drug distribution (poor tumor microcirculation) and below drug minimum effective concentration in tumor cells, as one of the pharmacokinetic/physiological categories, is one of common factors in treatment failure. In other words, the tumor poor microcirculation causing the poor drug distribution and below drug minimum effective concentration in tumor cells is directly associated with drug resistance during systemic treatment course. Meanwhile, it has been reported that cancer cell resistance may lead to failure of chemotherapy or targeted therapy due to mutations of cancer cells. Identifying the type of treatment resistance from cell-specific factors is extremely important for blood-borne therapies. Determining the type of treatment resistance as early as possible will give patients and clinicians the more opportunity to correct treatment strategies in a timely manner during the treatment process. Currently, there are no clinically available methods for distinguishing the type of drug resistance. In the present invention, the type of drug resistance is able to be identified during treatment or even before treatment, which can greatly improve current cancer treatment techniques.

4. A Computer Implemented Identification Method for Clinical Application

Herein, the computer software system is configured to process MRI raw data analyze the ability of fresh blood flowing through tumor area (equal to tumor drug distribution), visualize treatment response information on the infographic and assess patients' therapeutic information, identify the type of drug resistance. Also, the software system may be configured to run on different operation system platform and mobile devices in processing, recording, visualizing and sharing with clinicians for improving cancer treatment efficacy and reducing ineffective treatment.

The present invention may include four innovative aspects that perform the following functions: quantitatively evaluating ability of drug distribution characteristics of tumor via non-invasive Mill technology, designing a specific infographic platform to visualize cancer treatment response information (previous therapeutic response and future possible prognosis), establishing clinical applicable method for identifying the type of cancer drug resistances, and development of software for computerizing all the functions of the present invention on different operating system platforms.

The present invention provides a novel method for a clinician to timely identify the type of drug resistance during cancer treatment course. The clinical significance of the present invention is to provide a novel clinically applicable technique and method in clinical routine for identifying the type of drug resistance in a timely manner and assisting clinicians in conducting evidence-based cancer treatment. Cancer precision medicine is a method of providing the most suitable treatment method according to the characteristics of individual cancer. The present invention will provide a powerful tool for clinicians to monitor the characteristics of ineffective cancer treatment and modify the tumor microcirculation environment for the best treatment conditions in real time. It will make clinical cancer treatments more controllable and efficient. Precision medicine in cancer treatment is expected to become a mainstream medicine in the near future, the present invention will play a very important role in precision cancer treatment. The key of the present invention is to be able to identify the types of drug resistance in real time during tumor treatment, without being affected by changes in vascular permeability.

In some embodiments, a method of identifying tumor drug resistance for a particular solid tumor of a patient implemented by a clinical Mill scanner, an electronic device comprising a processor, a data input/output device, and a display input/output device, in which a MRI imaging protocol, a data processing algorithm, a cancer treatment response information diagram, and a method of drug resistance classification are applied to identification of the type of drug resistance during a tumor treatment scheme is provided. The method may include the steps of: when the patient is inhaling air, acquiring a first set of tumor multiple image slices and a serial of reference images of the particular solid tumor generated by dynamic contrast enhanced T2-weighted MR imaging technique with a data input/output device; when the patient is inhaling hyperoxic gas with increasing body blood oxyhemoglobin (HbO₂) concentration, acquiring a second set of tumor multiple image slices and a serial of enhanced images of the same particular solid tumor generated by same dynamic contrast enhanced T2-weighted MR imaging parameters with a data input/output device; calculating tumor region of interest (ROI) volume (V) based on intensity threshold of the dynamic contrast enhanced T2-weighted MR imaging data with the processor; computing a tumor volume change ratio (Vt %) based on a reference volume V₀ of the particular solid tumor with the processor; calculating a tumor all voxels' enhanced signal intensity (ΔSI) data with the processor; calculating tumor oxygenated perfusion percentage (OPP %) data based on a single threshold A with the processor; calculating a set of different thresholds of oxygenated perfusion percentage (OPP %) data and recording their location information with the processor; creating special different threshold value pseudo color maps with the processor; plotting OPP % data and Vt % data of the particular solid tumor on the cancer treatment response information diagram with the processor on the display input/output device; and identifying a type of drug resistance of the particular solid tumor based on analyzing the cancer treatment response information diagram with the processor on the display input/output device.

In further embodiments of the method, the oxygenated perfusion percentage data (OPP %) uses a threshold technique in analyzing dynamic contrast enhancement T2 weighted Mill signal for quantitatively measuring high oxyhemoglobin blood distribution of the particular solid tumor for evaluating tumor microcirculation and ability of drug distribution before and during tumor treatment, in which tumor microcirculation describes a pathophysiological phenomenon of the particular solid tumor, and in which drug distribution capabilities of the particular solid tumor describes the same pathophysiological phenomenon of particular solid tumor as tumor microcirculation.

In further embodiments, the method further includes plotting the oxygenated perfusion percentage data OPP % and displaying a reconstruction tumor oxygenated perfusion percentage (OPP %) pseudo color map using multiple threshold value during cancer treatment, wherein the pseudo-color maps of tumor oxygenation perfusion percentage (OPP %) with each measurement point during treatment are used to visualize and assess high OPP % and low OPP % areas of the particular solid tumor, wherein the tumor low OPP % areas are considered the hypoxic areas of the tumor and are targeted with relative high radiation dose for a Biologically-Guided Radiation Therapy.

In further embodiments, the method further includes the construction of a cancer treatment response information diagram, in which the diagram comprises two independent symmetrical OPP %-Vt % coordinate graphs composing a triangle structure, each OPP %-Vt % coordinate graph recording the different treatment response information, the two OPP %-Vt % coordinate graphs being mirrored and projected to each other.

In further embodiments, the method further includes integrating tumor volume change information (Vt %) and tumor oxygenated perfusion percentage information (OPP %) into one therapeutic response information point and displaying the point on one of sides OPP %-Vt % coordinate graph for evaluation of treatment response for a cancer therapy scheme.

In further embodiments, the method further includes plotting the oxygenated perfusion percentage (OPP %) and volume change ratio (Vt %) obtained from the same cancer therapy scheme plotting on the same side of the OPP %-Vt % coordinate graph for evaluation of tumor treatment response information.

In further embodiments of the method, the oxygenated perfusion percentage (OPP %) and volume change ratio (Vt %) obtained during current same cancer therapy scheme for the particular tumor is plotted on the left side of OPP %-Vt % coordinate graph, and in which the oxygenated perfusion percentage (OPP %) and volume change ratio (Vt %) data obtained from previous treatment records is plotted on the right side of OPP %-Vt % coordinate graph; wherein if the current therapy scheme is a combination of systemic treatment and irradiation treatment, the systemic treatment data is plotted on the left OPP %-Vt % coordinate graph, and the irradiation treatment data is plotted on the right OPP %-Vt % coordinate graph.

In further embodiments of the method, multiple sets of oxygenated perfusion percentage (OPP %) and volume change ratio (Vt %) from multiple measurement points during current cancer therapy scheme are plotted on the one side of OPP %-Vt % coordinate graph for evaluating tumor treatment response information and identifying the type of tumor drug resistance.

In further embodiments of the method, the current cancer treatment scheme is selected from the group consisting essentially of: systemic therapies (chemotherapy, molecular targeted therapy, immunotherapy, gene therapy, photodynamic therapy), local irradiation therapies (radiotherapy, hyperthermia therapy), and systemic therapies-local irradiation therapies combinations.

In further embodiments of the method, at least one previous cancer treatment response record is included the group consisting essentially of: systemic therapies (chemotherapy, molecular targeted therapy, immunotherapy, gene therapy, photodynamic therapy), local irradiation therapies (radiation therapy, hyperthermia therapy), systemic therapies-local irradiation therapies combinations.

In further embodiments, a method of using tumor oxygenated perfusion percentage (OPP %) and volume change ratio (Vt %) of a particular solid tumor for assisting evidence-based tumor precision medicine is provided, and the method may include: building up a OPP %-Vt % response database of different tumor responses to a treatment scheme, exploring the relationship between the measured tumor response information (oxygenated perfusion percentage (OPP %) and volume change ratio (Vt %) and their clinical outcomes of the treatment scheme; measuring multiple continuous measurements of the oxygenated perfusion percentage (OPP %) and a volume change ratio (Vt %) data with the treatment scheme; determining next treatment plan based on the measurement of individual tumor OPP % and Vt % data and the tumor OPP %-Vt % response database; and in which the steps of the method is performed by one or more electronic devices.

In further embodiments of the method, the oxygenated perfusion percentage (OPP %) and volume change ratio (Vt %) of current response and previous treatment response records are visualized on a cancer treatment response information diagram for reviewing, wherein the diagram comprises two independent symmetrical OPP %-Vt % coordination systems composing a triangle structure, and wherein both OPP %-Vt % coordinate graphs are mirrored and projected to each other.

In further embodiments, the method further includes plotting the oxygenated perfusion percentage (OPP %) and volume change ratio (Vt %) data obtained during same treatment scheme and plotting on the cancer treatment response information diagram for assisting evidence-based precision cancer treatment.

In further embodiments of the method, the oxygenated perfusion percentage (OPP %) and volume change ratio (Vt %) obtained during one cancer therapy modality for the particular solid tumor is plotted on left OPP %-Vt % coordinate graph, and wherein the data set of oxygenated perfusion percentage (OPP %) and volume change ratio Vt % obtained of another cancer therapy modality for the particular patient is plotted on the right OPP %-Vt % coordinate graph.

In some embodiments, a method of identifying the type of tumor drug resistance during a treatment scheme for a particular solid tumor, wherein it is applicable to all blood-borne therapies and local irradiation therapy except surgery is provided, and the method may include: completing multiple continuous measurements during the same treatment scheme, in which each oxygenated perfusion percentage (OPP %) and volume change ratio (Vt %) are obtained and plotted on an OPP %-Vt % coordinate graph of a cancer treatment response information diagram; continuously analyzing the multiple data sets and plotting them on the OPP %-Vt % coordinate graph as the treatment scheme progresses; retrospectively analyzing at least the latest two consecutive detection points of tumor OPP %-Vt % data, wherein if the consecutive measurements show that the tumor increases (Vt % is positive value) or the tumor continues to shrink (Vt % is negative value) and their difference is less than the critical value (3%), it is determined that the tumor is developing treatment resistance, and wherein identifying the type of tumor treatment resistance includes the following steps: if at least two consecutive measurements show that the tumor increases (Vt % is positive value) or the tumor continues to shrink (Vt % is negative value) and their difference is less than the critical value (3%) and the oxygenation perfusion percentage (OPP %) are all less than a critical value (5%), the resistance can be identified as the type of a pharmacological/physiological factors (low drug distribution and poor tumor microcirculation); and if at least two consecutive measurements show that the tumor increases (Vt % is positive value) or the tumor continues to shrink (Vt % is negative value) and their difference is less than the critical value (3%) and the oxygenation perfusion percentage (OPP %) data are all greater than a critical value (20%), the drug resistance can be identified as the type of a cell-specific factors; and during tumor treatment, drug resistance of the solid tumor and its type may change dynamically in response to different treatment schemes, and multiple measurements are necessary for timely and accurate identification of tumor drug resistance and its type.

In further embodiments of the method, the identification of drug resistance and its type is applicable to all “blood-borne therapies” modalities and “local irradiation therapy” modalities except surgical treatment. Measurement of the drug resistance during the treatment period can be arranged by the clinician according to the tumor response.

In further embodiments, the method of identification of drug resistance and its type is applicable to monitoring tumor normalization vasculature treatment via anti-angiogenic therapy, and wherein the parameter of oxygenated perfusion percentage (OPP %) is used to evaluate tumor vasculature remolding.

In further embodiments of the method, the standard of critical values for classifying the type of drug resistance is determined by the results of clinical statistics data, and the threshold values for classification is modified based on clinical data and are related to the tumor site and pathological stage.

### BRIEF DESCRIPTION OF THE DRAWINGS

Some embodiments of the present invention are illustrated as an example and are not limited by the figures of the accompanying drawings, in which like references may indicate similar elements and in which:

FIG. 1 depicts a block diagram of an example of a method for precision cancer treatment by identifying drug resistance according to various embodiments described herein.

FIG. 2 illustrates an example of a cancer treatment response information diagram according to various embodiments described herein.

FIG. 3A illustrates an example of breast cancer according to various embodiments described herein.

FIG. 3B illustrates an example cross sectional MR image of breast cancer according to various embodiments described herein.

FIG. 4A shows an example of a breast cancer tumor size measurement prior to an ineffective chemotherapy treatment situation. Shaded area indicates that the OPP % of area is greater than the threshold, and generating a gray map optionally uses only a single threshold.

FIG. 4B shows an example of a breast cancer tumor size measurement of the breast cancer tumor of FIG. 4A after a first an ineffective chemotherapy treatment course where OPP % is low and the tumor volume reduces small during treatment. Shaded area indicates that the OPP % of area is greater than the threshold, and generating a gray map optionally uses only a single threshold.

FIG. 4C shows an example of a breast cancer tumor size measurement of the breast cancer tumor of FIG. 4A after a second an ineffective chemotherapy treatment course where OPP % is low and the tumor volume reduces small during treatment. Shaded area indicates that the OPP % of area is greater than the threshold, and generating a gray map optionally uses only a single threshold.

FIG. 4D shows an example of a breast cancer tumor size measurement prior to an effective chemotherapy treatment situation. Shaded area indicates that the OPP % of area is greater than the threshold, and generating a gray map optionally uses only a single threshold.

FIG. 4E shows an example of a breast cancer tumor size measurement of the breast cancer tumor of FIG. 4D after a first effective chemotherapy treatment course where OPP % is high and tumor volume largely decreases during treatment. Shaded area indicates that the OPP % of area is greater than the threshold, and generating a gray map optionally uses only a single threshold.

FIG. 4F shows an example of a breast cancer tumor size measurement of the breast cancer tumor of FIG. 4D after a second effective chemotherapy treatment course where OPP % is high and tumor volume largely decreases during treatment. Shaded area indicates that the OPP % of area is greater than the threshold, and generating a gray map optionally uses only a single threshold.

FIG. 5 shows an example (FIGS. 4A-4C) of a breast cancer treatment response information diagram which describes an ineffective chemotherapy, treatment resistance caused by lower oxygenated perfusion percentage OPP % (inefficient drug distribution) and pharmacokinetic/physiological factor, cancer treatment according to various embodiments described herein.

FIG. 6 shows an example (FIGS. 4D-4F) of a cancer treatment response information diagram which describes high oxygenated perfusion percentage OPP % (high drug distribution) and an effective chemotherapy treatment according to various embodiments described herein.

FIG. 7 shows an example of a cancer treatment response information diagram which describes high oxygenated perfusion percentage OPP % (high drug distribution) and an ineffective chemotherapy or targeted therapy. The drug resistance is caused by cells-specific factors according to various embodiments described herein.

FIG. 8 shows an example of a cancer treatment response information diagram which describes an effective chemo-radiotherapy combination treatment according to various embodiments described herein.

FIG. 9 illustrates an example construction of a cancer treatment response information diagram according to various embodiments described herein.

FIG. 10 depicts an example of a block diagram of a server which may be used to perform one or more steps of the computer implemented a method for identifying the type of drug resistance in cancer treatment according to various embodiments described herein.

FIG. 11 illustrates an example of a block diagram of an electronic device which may be used to perform one or more steps of the computer implemented identification method and to generate a cancer treatment response information diagram according to various embodiments described herein.

FIG. 12 shows an illustrative example of some of the components and computer implemented methods which may be found in a cancer therapy treatment resistance identification system according to various embodiments described herein.

FIG. 13 depicts a block diagram illustrating some applications of a cancer therapy treatment resistance identification system which may function as software rules engines according to various embodiments described herein.

FIG. 14 illustrates a block diagram of an example of a method for generating an estimation of how to identify the type of therapy resistance of a cancer of a particular patient according to various embodiments described herein.

FIG. 15A shows a table providing some example critical values for evaluating tumor low drug distribution as a factor for determining resistance of a patient's cancer according to various embodiments described herein.

FIG. 15B shows a table providing some example critical values for determining resistance of a patient's cancer to anti-angiogenic therapy according to various embodiments described herein.

FIG. 15C shows a table providing some example critical values for evaluating tumor cells-specific factors for determining resistance of a patient's cancer according to various embodiments described herein.

FIG. 16 illustrates a block diagram of an example of a method for precision cancer treatment by identifying drug resistance according to various embodiments described herein.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

As used herein, the term “cancer” or “tumor” refers to the mammalian, such as a human, solid tumor or solid cancer in any site which can be detected by Magnetic Resonance Imaging (MRI).

As used herein, the term “Flow and Oxygenation Dependent (FLOOD) MM” and “T2-weighted MR imaging technique” refers to the clinical conventional 1.5T or 3T MM scanner which is used to non-invasively detect tumor variation of hemoglobin concentration information.

As used herein, the term “image slices” or “slices” refers to the patient's tumor is first divided into a set of slices and each slice is composed of a voxel matrix during an MRI procedure. Each slice shows the physiological information within each layer of the tumor. The thickness of slice and size of the voxel are related to the performance of Mill scanner and MR imaging pulse sequence.

As used herein, the term “fresh blood” and “oxygenated blood perfusion” and “high oxyhemoglobin blood perfusion” refers to the high oxyhemoglobin concentration blood which comes from tumor arterial of vascular system. When fresh blood flows through the tumor area, gas exchange occurs between the vessels and the surrounding tissues of the blood vessels. The oxyhemoglobin concentration in the vessel should gradually decrease and reach the oxygen equilibrium with the surrounding tissues.

As used herein, the term “ability of oxygenated blood” refers to the capability of artificially generated high oxyhemoglobin blood flowing through tumor. Here, this artificial temporary increase in blood oxygen saturation depends on individual patient's lung function by breathing hyperoxic gas. The variation of deoxyhemoglobin concentration dHbO₂ in tumor area can reflect the local blood circulation status of the tumor by compared with before and after artificial changing blood oxygen saturation. During this process, the largest change in dHbO₂ is located in the arterial parts; the smallest or no change in dHbO₂ is located in the venous parts. Here, the dHbO₂ as an endogenous contrast agent is used to detect the local blood microcirculation of tumor via the dynamic contrast enhancement T2-weighted MM technique.

As used herein, the term “tumor microcirculation” refers to the capability of blood carrying oxygen, nutrition molecular flowing through tumor region. Here, the measurement approach of the present invention is to measure high oxyhemoglobin concentration blood flow through tumor area for evaluating the tumor microcirculation, which result can also simulate the ability of drug-carrying blood to distribute in tumor area. The state of tumor microcirculation can be dynamically changed with the change of tumor volume.

As used herein, the term “ability of drug distribution” refers to the potential capability of drug delivery and distribution inside tumor. Here, it can be measured by analyzing ability of oxygenated perfusion because the drugs transportation and delivery in the plasma is similar to that of carrying oxygen transportation and delivery from arterial to capillaries to vein inside the tumor. Due to the special measurement method of the present invention, tumor microcirculation and ability of drug distribution have the similarity in cancer pathophysiology here. The relative value is used a unit for evaluating the ability of drug distribution in tumor. The higher percentage of fresh blood flowing in tumor area, the better ability of drug delivery and distribution in tumor area.

As used herein, the term “vascular permeability”, “capillary permeability”, “microvascular permeability”, or “permeability” refers to the ability of a blood vessel wall to allow for the flow of small molecules (drugs, particles, cells, nutrients, water, ions) or even whole cells (lymphocytes) in and out of the vessel. Blood vessel walls are lined by a single layer of endothelial cells. The gaps between endothelial cells (cell junctions) are strictly regulated depending on the type and physiological state of the tissue. Vascular permeability allows drugs/agents of blood-born therapies to penetrate blood vessels into the extravascular extracellular space (EES). Clinically, the permeability of tumor blood vessels is higher than that of normal tissues, which can change drastically during cancer treatment.

As used herein, the term “oxygenated perfusion percentage” or “OPP %” refers to the ability of high oxyhemoglobin blood flowing through tumor area. Here, it is a parameter to evaluate the ability of tumor oxygenated blood perfusion using threshold technique, which is similar to the ability of drug distribution inside tumor. It is a prognostic parameter for assessing future possible therapeutic response.

As used herein, the term “volume change ratio” or “Vt %” refers to the varication of tumor volume comparing with the tumor volume of before first treatment (reference volume), the negative value means tumor shrinkage, the positive value means tumor volume increase.

As used herein, the term “therapeutic resistance” or “treatment resistance” or “drug resistance” refers to the resistance in cancer therapies, which may happen to most cancer patients. Herein, it can be defined as the multiple consecutive measurements of tumor volume only shrink smaller than 3% during treatment or the tumor volume are increased.

As used herein, the term “type of drug resistance” refers to the characteristic of treatment resistance, which can be divided into two broad categories: cells-specific factors and pharmacological/physiological factors. Drug resistance of cells-specific factors may have several possible reasons: Some of the cancer cells that are not killed by the therapeutic drug mutate and become resistant to the drug. Once they multiply, there may be more resistant cells than cells that are sensitive to the therapy. A cancer cell may produce hundreds of copies of a particular gene. This gene triggers an overproduction of protein that renders the anticancer drug ineffective. Cancer cells may pump the drug out of the cell as fast as it is going in using a molecule called p-glycoprotein. Cancer cells may stop taking in the drugs because the protein that transports the drug across the cell wall stops working. The cancer cells may learn how to repair the DNA breaks caused by some anti-cancer drugs. Cancer cells may develop a mechanism that inactivates the drug. Even a tumor with good drug distribution/concentration may still have drug resistance due to cancer cells-specific factors. The drug resistance of pharmacological/physiological factors may be caused by below drug minimum effective concentration in tumor cells. The poor drug delivery and distribution in tumor regions related to tumor microcirculation is one of the factors in pharmacological/physiological category.

As used herein, the term “blood-borne therapies” or “systemic therapy” or “systemic treatment” refers to the systemic therapies. Here, the drugs/agents of systemic therapies are transported by blood that spread throughout the body to treat cancer cells. They include chemotherapy, hormonal therapy, targeted therapy, immunotherapy, gene therapy, and photodynamic therapy.

As used herein, the term “irradiation therapy” or “local irradiation therapy” refers to the local therapies with irradiating energy or rays to damage cancer cellular structure. Here, it includes the photon, electron, proton radiation therapy, thermotherapy.

As used herein, the term “precision medicine in cancer treatment” and “precision cancer treatment” refers to an emerging method of providing the most suitable treatment method according to the characteristics of cancer. Here, the precision cancer treatment specifically refers to all cancer therapies clinically except surgical treatment.

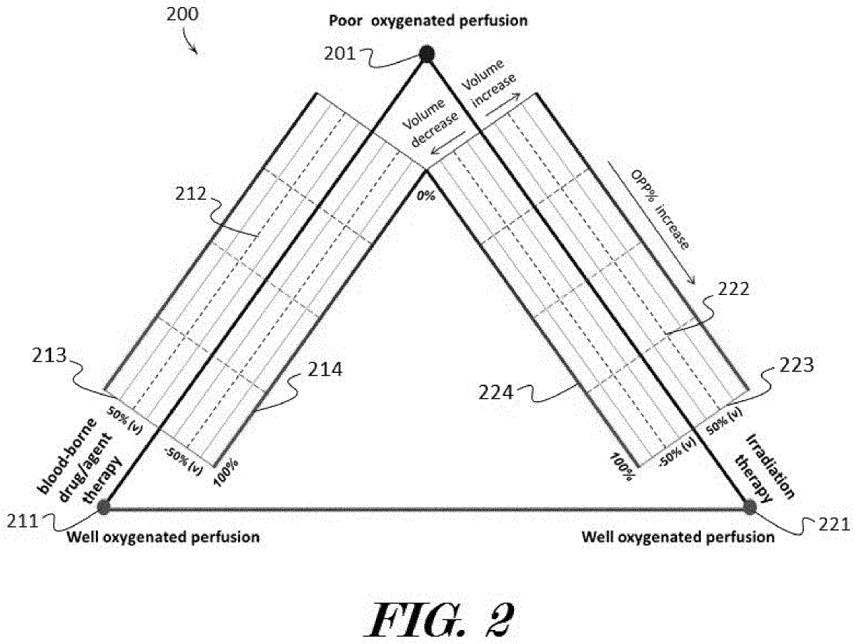

As used herein, the term “cancer treatment response diagram”, or “cancer treatment response information diagram”, or “diagram”, or “cancer treatment infographic” or “OPP %-Vt % coordinate graph” refers to the integrating the tumor volume change ratio Vt % and oxygenated perfusion percentage OPP % as one therapeutic response information point on the cancer treatment response information diagram as shown in FIGS. 2 and 5-9. One cancer treatment response information diagram may have multiple different treatment response information points during different periods of cancer treatment.

As used herein, the term “computer” refers to a machine, apparatus, or device that is capable of accepting and performing logic operations from software code. The term “application”, “software”, “software code” or “computer software” refers to any set of instructions operable to cause a computer to perform an operation. Software code may be operated on by a “rules engine” or processor. Thus, the methods and systems of the present invention may be performed by a computer or computing device having a processor based on instructions received by computer applications and software.

As used herein, the term “electronic device” is a type of computer or computing device comprising circuitry and configured to generally perform functions such as recording audio, photos, and videos; displaying or reproducing audio, photos, and videos; storing, retrieving, or manipulation of electronic data; providing electrical communications and network connectivity; or any other similar function. Non-limiting examples of electronic devices include: personal computers (PCs), workstations, laptops, tablet PCs including the iPad, cell phones including iOS phones made by Apple Inc., Android OS phones, Microsoft OS phones, Blackberry phones, digital music players, or any electronic device capable of running computer software and displaying information to a user, memory cards, other memory storage devices, digital cameras, external battery packs, external charging devices, and the like. Certain types of electronic devices which are portable and easily carried by a person from one location to another may sometimes be referred to as a “portable electronic device” or “portable device”. Some non-limiting examples of portable devices include: cell phones, smartphones, tablet computers, laptop computers, and wearable computers such as Apple Watch, other smartwatches, Fitbit, other wearable fitness trackers, Google Glasses, and the like.

As used herein, the term “user device” or sometimes “electronic device” or just “device” is a type of computer or computing device generally operated by a person or user of the system. In some embodiments, a user device is a smartphone or computer configured to receive and transmit data to a server or other electronic device which may be operated locally or in the cloud. Non-limiting examples of user devices include: personal computers (PCs), workstations, laptops, tablet PCs including the iPad, cell phones including iOS phones made by Apple Inc., Android OS phones, Microsoft OS phones, Blackberry phones, or generally any electronic device capable of running computer software and displaying information to a user. Certain types of user devices which are portable and easily carried by a person from one location to another may sometimes be referred to as a “mobile device” or “portable device”. Some non-limiting examples of mobile devices include: cell phones, smartphones, tablet computers, laptop computers, wearable computers such as Apple Watch, other smartwatches, Fitbit, other wearable fitness trackers, Google Glasses, and the like.

As used herein, the term “computer readable medium” refers to any medium that participates in providing instructions to the processor for execution. A computer readable medium may take many forms, including but not limited to, non-volatile media, volatile media, and transmission media. Non-volatile media includes, for example, optical, magnetic disks, and magneto-optical disks, such as the hard disk or the removable media drive. Volatile media includes dynamic memory, such as the main memory. Transmission media includes coaxial cables, copper wire and fiber optics, including the wires that make up the bus. Transmission media may also take the form of acoustic or light waves, such as those generated during radio wave and infrared data communications.

As used herein, the term “data network” or “network” shall mean an infrastructure capable of connecting two or more computers such as user devices either using wires or wirelessly allowing them to transmit and receive data. Non-limiting examples of data networks may include the PACS (picture archiving and communication system) internet or wireless networks or (i.e. a “wireless network”) which may include Wifi and cellular networks. These networks may use any security protocol suitable for securing patient health information and other protected information. For example, a network may include a local area network (LAN), a wide area network (WAN) (e.g., the Internet), a mobile relay network, a metropolitan area network (MAN), an ad hoc network, a telephone network (e.g., a Public Switched Telephone Network (PSTN)), a cellular network, or a voice-over-IP (VoIP) network.

As used herein, the term “database” shall generally mean a digital collection of data or information. All Mill images raw data is stored on a file system in DICOM format. The present invention uses novel methods and processes to store, link, and modify information such digital images and videos and user profile information. For the purposes of the present disclosure, a database may be stored on a remote server and accessed by a user device through the internet (i.e., the database is in the cloud) or alternatively in some embodiments the database may be stored on the user device or remote computer itself (i.e., local storage). A “data store” as used herein may contain or comprise a database (i.e. information and data from a database may be recorded into a medium on a data store).

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

The present disclosure is to be considered as an exemplification of the invention, and is not intended to limit the invention to the specific embodiments illustrated by the figures or description below.

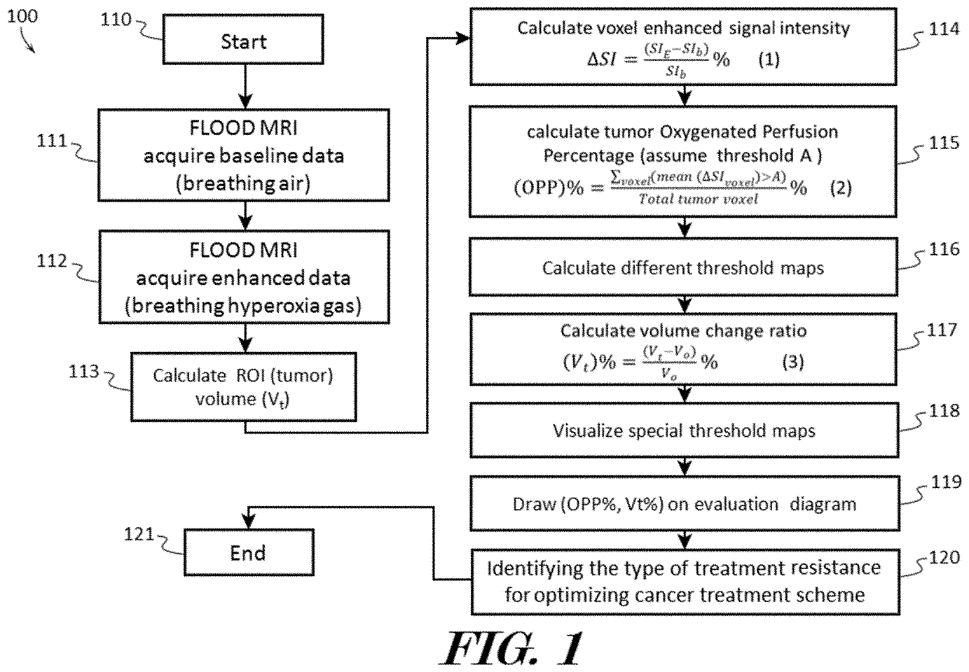

The present invention will now be described and computerized by example, algorithms, and through referencing the appended figures representing preferred and alternative embodiments. FIG. 1 illustrates a block diagram of an example of a computer implemented the identification method (“the method”) 100 according to various embodiments. FIGS. 2 and 5-9 illustrate cancer treatment response information diagrams 200 for identification of the type of drug resistance. In the following description, for purposes of explanation, numerous specific details are set forth in order to provide a thorough understanding of the present invention. It will be evident, however, to one skilled in the art that the present invention may be practiced without these specific details.

Quantitatively Evaluating Drug Distribution Characteristics of Tumor

The vasculature, composed of vessels of different morphology and function, distributes blood to all tissues and maintains physiological tissue homeostasis. In pathophysiology, the tumor vasculature is often affected by, and engaged in, the disease process. This may result in excessive formation of new, unstable, and hyperpermeable vessels with poor blood flow, which further promotes hypoxia and disease propagation. Microcirculation is the circulation of the blood in the smallest blood vessels, present in the vasculature embedded within organ tissues. The main functions of blood in the microcirculation are the delivery of oxygen (O₂), nutrients, drug/agent and the removal of carbon dioxide (CO₂). The vascular system of tumor is totally different from that of normal tissue, which usually is abnormal vascular structure and high vascular permeability in the tumor region. The cancer mass without blood circulation can grew to 1-2 mm³ in diameter and then stopped, but grew beyond 2 mm³ when placed in an area where angiogenesis was possible. Tumor angiogenesis allows for supply of oxygen, nutrients, growth factors, and tumor dissemination to distant sites. Abnormal tumor vasculature typically lacks hierarchical structure and is composed of immature differentiated and undifferentiated vessels with increased permeability. The undifferentiated vessels frequently present with either collapsed or an absent lumen. For a blood-borne cancer therapeutic agent to be effective, the drug/agent must cross the blood vessel wall to reach cancer cells in adequate quantities, and it must require to overcome the resistance conferred by the local microenvironment around cancer cells. The distribution of drugs in tumor area must go through two important stages: drug transport in capillaries and drug/agent penetration from capillaries to the extravascular extracellular space (EES), that is, the tumor's microcirculation and microvascular permeability. The microvascular permeability is closely related to the structure of the angiogenesis, the size of the drug/agent and its concentration in plasma. The concentration of the drug in the plasma decays over time, and the permeability capacity may change dynamically and drastically due to treatment response. For evaluation of the tumor drug distribution, it is necessary to consider avoiding the influence of vascular permeability. Our evaluation of drug distribution can only focus on evaluating the ability of intratumoral drug transportation in tumor capillaries, which has the advantage of avoiding the influence of unpredictable physiological factors on capillary permeability. This is the reason why the present invention adopts the special new imaging protocol technology of T2-weighted Mill and data processing algorithms.

The delivery of oxygen to tumor cells to maintain their metabolism depends on the transportation of high oxyhemoglobin blood between the body blood circulation system and local tumor microcirculation. Blood with a high oxyhemoglobin (HbO₂) concentration flow from the tumor artery to the capillaries, and then venules, where gas exchange between blood and surrounding tissue. The gas exchange process shows that the concentration of HbO₂ in the blood gradually decreases, while the concentration of deoxyhemoglobin (dHbO₂) in the vessel gradually increases. They eventually reach the oxygen equilibrium between vein side and surround tissue. The condition of gas exchange is that there must be an area through which high HbO₂ blood flows in. Similarly, the oxygen delivery process in tumor area is similar to drug distribution process that the drug in plasma is delivered to the tumor area also depends on the flow of arterial blood through the tumor area. If the capillary permeability is assumed to be unchanged, arterial blood with prescription drug concentration plasma flows through the tumor area, the larger the tumor area that passes through, the better drug distribution in the tumor. It is closely related to the clinical blood-born therapies effects. Differences in individual tumor angiogenesis can directly lead to individual differences in the ability of drug distribution, that is directly associated with the drug resistance of tumor. Here, to evaluate tumor drug distribution can be achieved by analyzing the ability of the high HbO₂ blood flowing through tumor area.

Contrast enhanced Mill technology has been widely used in clinical routine for tumor diagnosis. Solid tumors have higher vascular permeability than normal tissues. When intravenous injection of gadolinium-diethylenetriamine pentaacetic acid (Gd-DTPA) and part of exogenous contrast agent leaking out of vessels and staying extravascular extracellular space (EES), using T1-weighted Mill pulse sequence, the intensity of the Mill T1-weighted signal in the area of the high exogenous contrast agent area is significantly higher than that of the normal tissue. This high signal intensity causing by tumor high vascular permeability is applied to diagnose solid tumor. This clinical T1-weighted Dynamic Contrast Enhancement (DCE) MRI technique has been widely used to identify the size and location of tumors for solid tumor diagnosing and screening. More precisely, the high permeability of tumor capillaries is a key of cancer imaging principle in current clinical MRI diagnosis of tumors for ensuring high detection sensitivity and accuracy. Under the same conditions of local blood flow in tumor, the permeability difference of tumor capillaries may determine the difference of the enhanced T1 signal intensity of MM. Unfortunately, clinical studies have shown that the tumor vascular permeability will change drastically during tumor treatment. Decreased vascular drug permeability will directly result in decreased drug accumulation in the extravascular extracellular space (EES) of tumor region, which will affect the result of T1-weighted MM measurement during treatment. For example, antiangiogenic therapies can decrease the tumor vascular permeability. Clinical studies now show that vascular permeability may be actively manipulated to improve blood-borne treatment, because reducing vascular permeability significantly limits the ability of drugs to access tumor cells. Although how to manipulate vascular permeability belongs to the clinical field of cancer therapy, the impact of permeability should be avoided during identifying the type of drug resistance. Due to the randomness of vascular permeability changes, the identification of drug resistance will focus on the delivery and distribution of drugs in the tumor microcirculation.

As the treatment causes changes in tumor microvascular permeability, it will directly cause the false information in T1-weighted Mill signals, which cannot be used to assess tumor biological information (especially tumor blood perfusion or tumor microcirculation). A large number of clinical research reports have confirmed this phenomenon that T1-weighted Mill has error to monitor tumor biological information during treatment. Currently, the clinical examination of Dynamic Contrast Enhancement (DCE) T1-weighted Mill has to be arranged after at least two or three months completing clinical treatment. The contrast enhancement T1-weighted Mill technology using exogenous contrast agents is influenced by treatment that affects vascular permeability, such as antiangiogenic therapies for tumor vascular normalization. In order to minimize the measurement errors causing by tumor microvascular permeability, some Mill pre-clinical studies on tumor blood perfusion have been reported on the use of injecting extrinsic special exogenous macromolecular contrast agents (large size contrast molecular) to minimize the impact of vascular permeability. However, the FDA has not approved the clinical application of this kind of contrast agent because it may cause serious clinical problems. Although CT has been widely used to monitor the tumor volume during treatment, due to the influence of permeability, there are also errors in the use of CT and exogenous contrast agent technology to evaluate the physiological information of tumor microcirculation or blood perfusion. In short, the detection of tumor physiological information such as blood perfusion and microvascular function through exogenous contrast agents of MM technology will be greatly affected by changes in vascular permeability during the treatment process. However, tumor vascular permeability will change dramatically in response to different treatment drugs and options. This reveals the limitations of Mill exogenous contrast agent technology that cannot accurately evaluate the physiological response of tumors during treatment.

If the effect of drugs' size and concentration in plasma on vascular permeability being ignored, the more effective the blood oxygen delivery in the tumor area, the better the ability of the drug delivery area, and the better the drug distribution ability in the same area. Once the tumor area flows through the blood with high oxygenated hemoglobin concentration, it may also indicate that the oxygen partial pressure pO₂ in the same area is relatively high. It has been clinically proven that the high pO₂ value of tumors is closely related to the better prognostic effect. However, the tumor local oxygen pressure pO₂ is also highly related to other factors, such as blood flow, blood pH, glucose levels, and cancer cells' metabolism. There is technologic difficulty to measure local PO₂ map of tumor in the clinical setting. Similarly, PET/CT technology by injecting radiotracers is hard to measure the capacity of drug distribution in the clinical routine.

In the present invention, an improved Flow and Oxygenation Dependent (FLOOD) T2-weighted MM imaging technology is introduced for clinically non-invasive detection of the tumor oxygenated blood flow. This technology can be easily applied to 1.5 T or 3T MRI clinical scanner systems. As a new imaging protocol introduces in the present invention, a serial of reference images with multiple slices of tumor are taken as reference images using the T2 weighted MM pulse sequence imaging technique when the patient inhales air first, then artificially causing a high concentration of oxyhemoglobin (HbO₂) to circulate in the body via breathing a hyperoxic gas for a while, a long serial of images with same slices of tumor is taken using the same T2-weighted MRI pulse sequence with the same position and imaging parameters, finally patient inhales air again after MRI scanning. When the artificial high hemoglobin (HbO₂) blood in the tumor flows from arterial to the vein, the HbO₂ in the blood gradually decreases, while dHbO₂ gradually increases until it reaches an equilibrium of the partial pressure of oxygen (pO₂) around the tissue. Since dHbO₂ is paramagnetic, and HbO₂ is non-paramagnetic, the conversion from HbO₂ to dHbO₂ in the blood will produce MR T2 signal gain, and the magnitude of MR enhancement is positively correlated with the magnitude of the change of oxyhemoglobin concentration. Actually, MR T2 signal gain is related to variation of blood hemoglobin concentration, blood flow velocity and flowing direction to MM static magnetic field B₀.

MRI studies have proved that the contribution of MR T2 signal intensity caused by changes in blood oxyhemoglobin concentration will exceed 10 times the change in blood flow velocity. By applying a special data processing algorithm to strictly compare the response of the same voxel to breathing air and high-concentration oxygen gas at the same position, the influence of blood flow velocity and flowing direction on the T2 signal can be minimized. This endogenous contrast agent enhancement MM technology for measuring fresh blood distribution is more sensitive than fitting tumor hypoxia R2*(1/T2*) map technology (this MRI T2* fitting map technology is still under exploration) to assess the tumor hypoxic map. Physiologically, there is no correlation between changes in oxyhemoglobin concentration and vascular permeability. One of unique advantages of the endogenous contrast agent dHbO₂ is that the exchange of oxygen molecules in the blood does not depend on the vascular permeability of the tumor, and oxygen molecules can directly rapidly diffuse across the blood vessel wall cells to the surrounding tissues. Compared with other MRI technologies, the T2-weighted MRI imaging principle of the present invention shows unique capabilities.

The ability of fresh (high oxyhemoglobin concentration) blood to flow through the tumor has a physiological process similar to tumor drug delivery and distribution. In the present invention, to evaluate the ability of tumor drug distribution can be translated into quantitatively analyzing the ability of fresh (high oxyhemoglobin) blood flowing through the tumor area during treatment process.

The MRI data processing will be focused on the tumor region of interest (ROI). The relative signal intensity of the tumor ROI will be processed on voxel-by-voxel basis. When the patient breathes air, the average value of the MR signal intensity of multiple scans for each voxel is calculated and defined as the baseline or reference value for that voxel. When the patient inhales high-concentration oxygen, the MR relative signal intensity of each voxel continuous scan is calculated based on the reference value and the enhanced signal of each scan. This processing algorithm can also minimize the influence of blood flow changes on T2-weighted MRI signal and highlight the contribution of oxygen saturation changes to the MR signal. In some embodiments, the following equation is used to calculate the relative signal intensity (ΔSI) of the tumor on a voxel-by-voxel basis:

$$Δ SI = \frac{( S {I}_{E} - S {I}_{b} )}{S {I}_{b}} \% \qquad ( 1 )$$

Where, SI_{E} refers to the voxel's enhanced signal intensity of each scan during breathing hyperoxia gas; SI_{b} is defined as the average of signal intensity of the baseline value in same voxel breathing air. The high relative signal intensity (ΔSI) tumor area may represent the region with a high T2 contrast enhancing effect. That is, the higher the relative signal strength of the voxel, the greater the change in oxyhemoglobin concentration during the inhalation of hyperoxia gas, which indicates that voxel has perfused by fresh oxygenated blood.

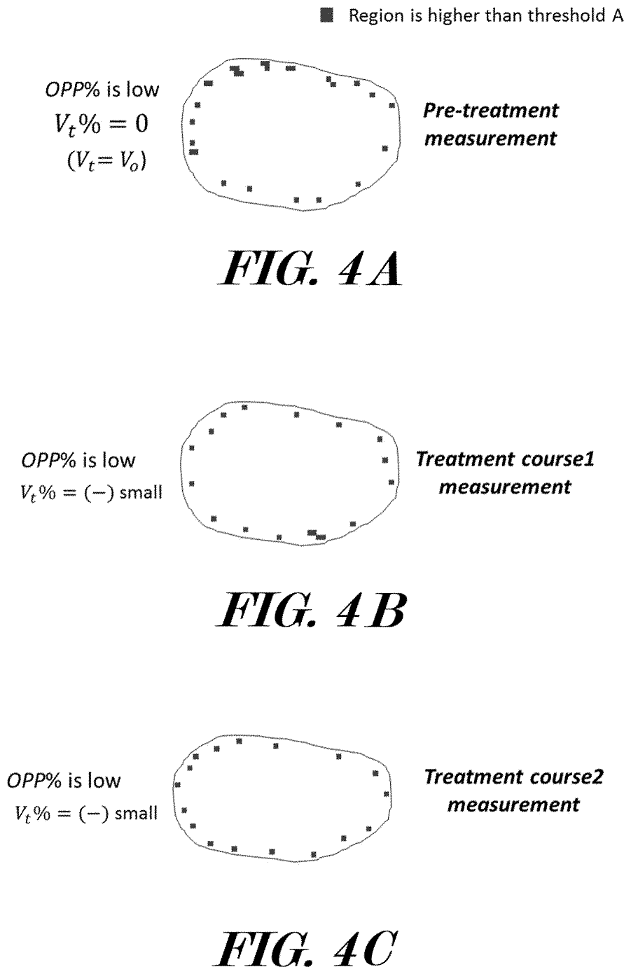

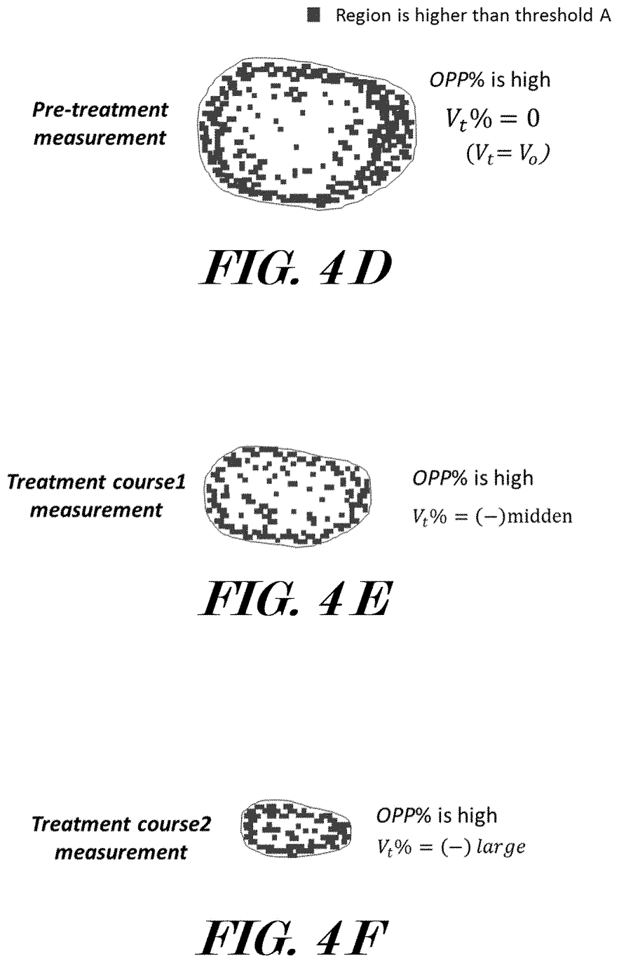

In order to quantitatively evaluate the ability of the fresh blood flowing through whole tumor area, the threshold technique was selected to classify all voxel of the tumor. If the relative signal intensity (ΔSI) of the voxel is higher than a threshold A, it means, there is fresh oxygenated blood flowing through in that voxel. The more voxels with the relative signal intensity (ΔSI) being above the threshold A, the greater the proportion of the tumor area with fresh blood flowing through. The accumulation of all voxels with a relative signal strength (ΔSI) above the threshold A can be used to calculate a percentage of volume whose ΔSI of voxel are above the threshold A, so called oxygenated perfusion percentage (OPP %). The OPP % can quantitatively represent the parameters of ability of fresh oxyhemoglobin blood flowing through tumor area, which may also indicate the ability of drug distribution inside tumor and the state of tumor microcirculation. In preferred embodiments, oxygenated perfusion percentage (OPP %) is to use threshold technique in processing contrast enhancement T2 weighted MM signal intensity for quantitatively evaluating ability of tumor drug distribution before or during the course of treatment. High (OPP %) means that more area of the tumor flowing through high oxyhemoglobin blood, which represents better drug/agent/oxygen delivery and distribution. The oxygenated perfusion percentage (OPP %) of tumor can be quantified by following equation:

$$( OPP ) \% = \frac{{Σ}_{voxel} ( mean ( Δ {SI}_{voxel} ) > A )}{Total tumor voxel} \% \qquad ( 2 )$$

Where, ΔSI refers to each voxel's relative signal intensity during the patient's hyperoxia gas inhalation; the A refers to a threshold value for classification of enhanced relative signal intensity. Threshold technology is a general scientific analysis method to classify different data. Here, the value of the A should be related to the MR imaging pulse sequence, TR/TE time, thickness of slices, the strength of the magnet of the clinical scanner, the sensitivity of the coil, the location of the cancer, and so on. For example, it can be assumed a standard threshold 10% for 1.5T and 15% for 3T Mill scanner. The lower OPP % may represent lower (inefficient) drug/agents' distribution and poor tumor microcirculation which may be associated with ineffective treatment outcomes (FIGS. 4A-4C). Conversely, the higher OPP % of tumor may be, the better drug/agent's distribution and better microcirculation which may be associated with effective treatment outcomes (FIGS. 4D-4F).

In the present invention, the new MRI imaging protocol technology and data processing algorithm are particularly applicable to evaluate the distribution of high oxyhemoglobin blood flow through tumor area. Theoretically, the impact of the varication in vascular permeability can be totally ignored. It shows great advantages for assessing tumor drug distribution during treatment. Also, inhaling hyperoxia gas (oxygen) to produce endogenous contrast effect does not produce any side effects on the human body. It will be the first time to allow us evaluating the ability of individual tumor drug distribution before or during treatment, which is the unique advantage of the present invention.

Designing a specific infographic (“the diagram”) and visualizing tumor treatment response information

Usually, tumor volume responses to effective treatment may be clinically delayed for a few days or weeks. As an important parameter, tumor volume has been a standard to assess previous effects during treatment. Traditional X-ray, ultrasound, CT, and even doctor's palpation techniques are used to check the changes of tumor volume during treatment. Although tumor volume delays in response to treatment are common behaviors, volume change information remains an objectively valuable parameter in assessing previous treatments. Here, the tumor volume is calculated by the accumulation of voxels in the tumor ROI of each measurement based on the T2-weighted MRI scanning. The tumor volume before first treatment is used as the tumor reference volume, and the tumor volume change ratio Vt % of each measurement is calculated based on the current measured volume and the reference volume value.

The tumor volume before first treatment V₀ is defined as reference of volume. Each measurement of tumor volume during treatment can be compared with reference value and calculated:

$$( {V}_{t} ) \% = \frac{( {V}_{t} - {V}_{o} )}{{V}_{o}} \% \qquad ( 3 )$$

Where, V₀ is the tumor reference volume before first treatment; Vt is the measured volume of tumor during treatment. The first measurement (before first treatment), Vt %=0; When the tumor volume shrinks, the Vt % shows a negative value; If the tumor volume increases during treatment, the Vt % shows a positive value; If the tumor completely responds to treatment and disappears, the Vt %=−100%.

Usually, changes in tumor volume are not sufficient to timely reflect the tumor future response to next treatment. There are many uncertainties in treatment. For example, tumor atrophy or swelling in different location of tumor may lead to changes in microcirculation patterns and their internal hemodynamics, which may directly lead to change in drug distribution and resistance in next treatment. To monitor the dynamic change of tumor microcirculation or ability of drug distribution during treatment process shows a significant meaning in cancer treatment. In fact, clinicians are eager to know two different types of treatment response information immediately during treatment: the previous treatment result Vt %, and the future possible response OPP % (prognosis information). In the present invention, these two parameters have been used as a therapeutic response information point on a particular coordinate system. More importantly, it can provide clinicians the opportunity to visualize previous treatment effect and assess tumor possible outcomes on the one infographic. By analyzing tumor response information, it can help clinicians to adjust treatment strategy, optimize therapy plan, and achieve evidence-based cancer treatment. Another advantage of the two-dimensional response data style can be further used for identifying the type of resistance factor during treatment. infographic.

Here, two parameters of oxygenated perfusion percentage OPP % and tumor volume change ratio Vt % are both relative values based on simplified and intuitive calculation methods. Its purpose is to simplify expression and infinite markable range on tumor information diagram. It is also a clinical habitual usage.

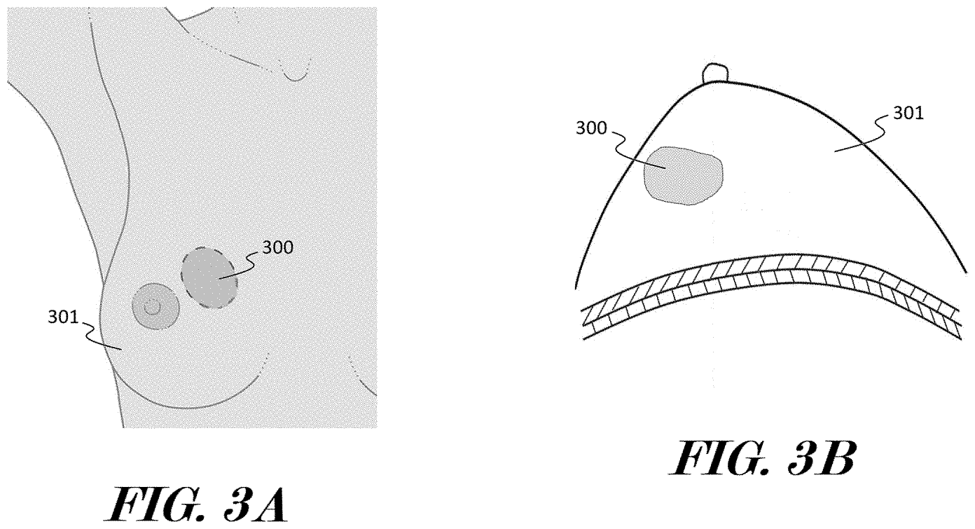

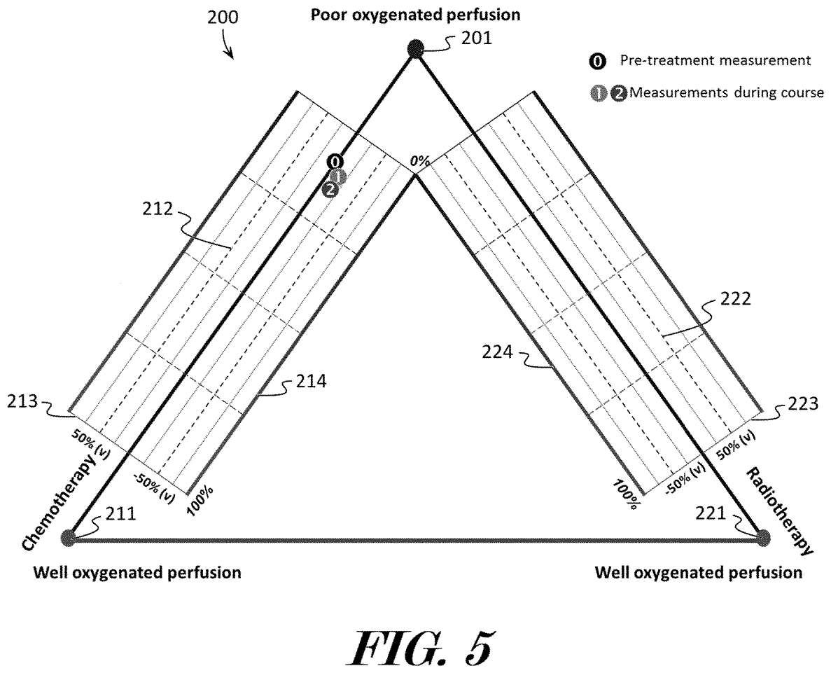

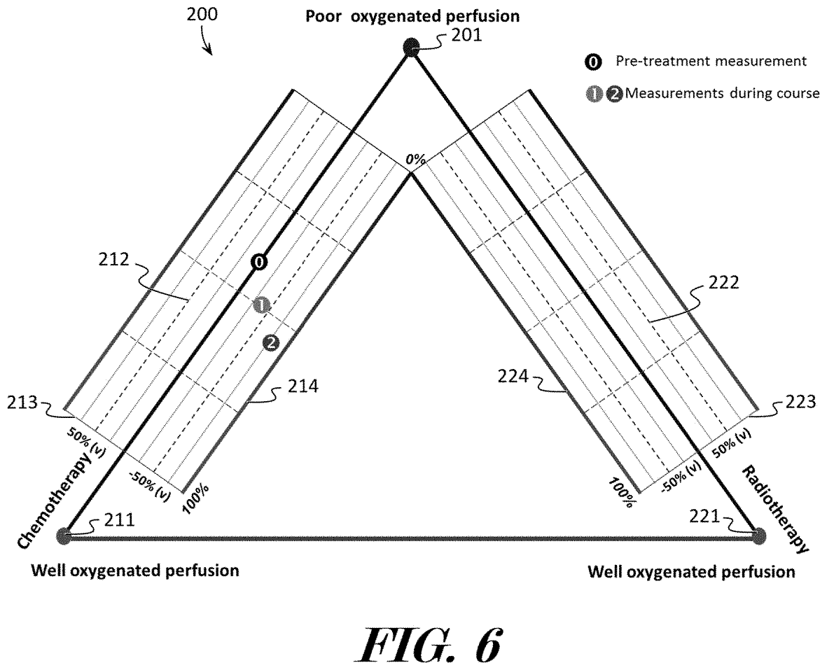

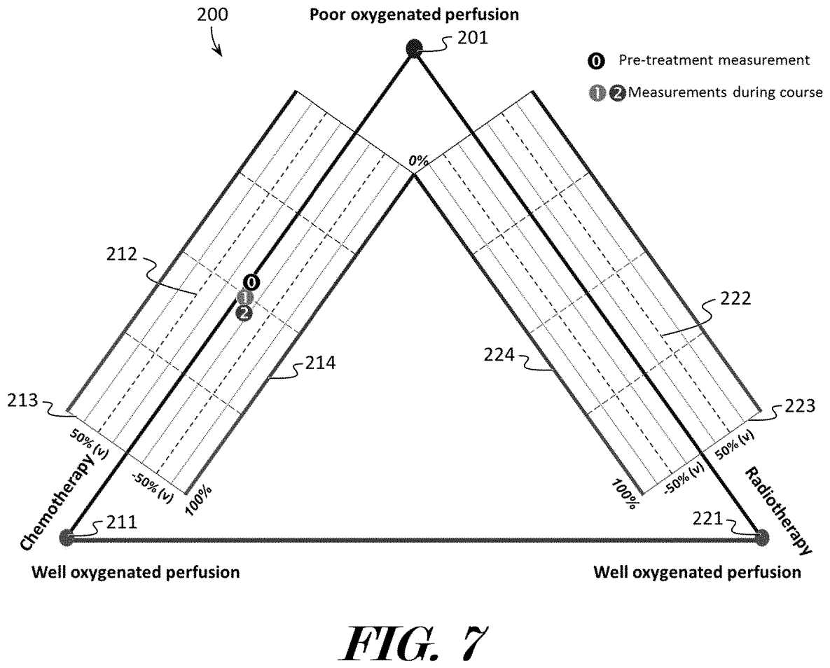

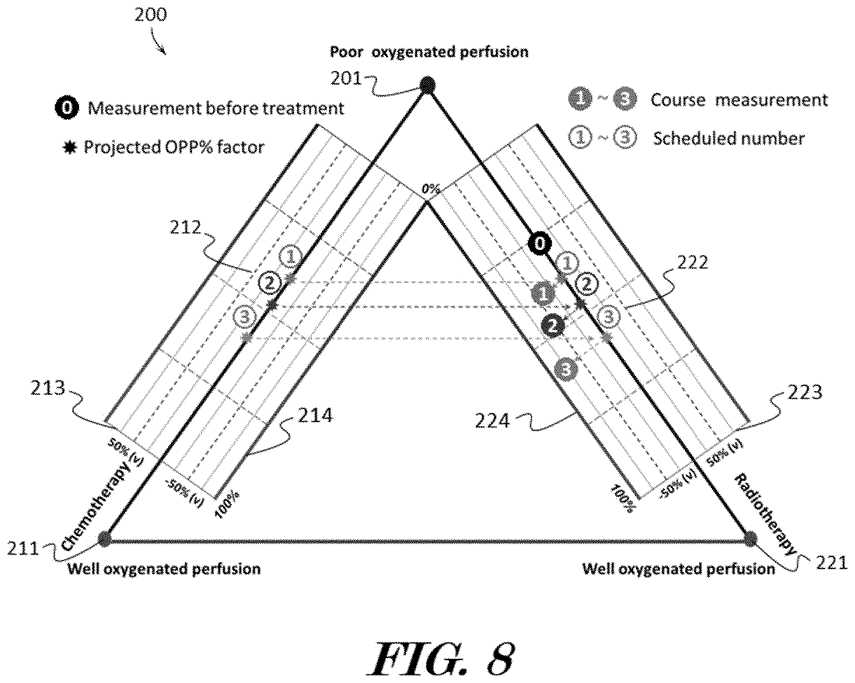

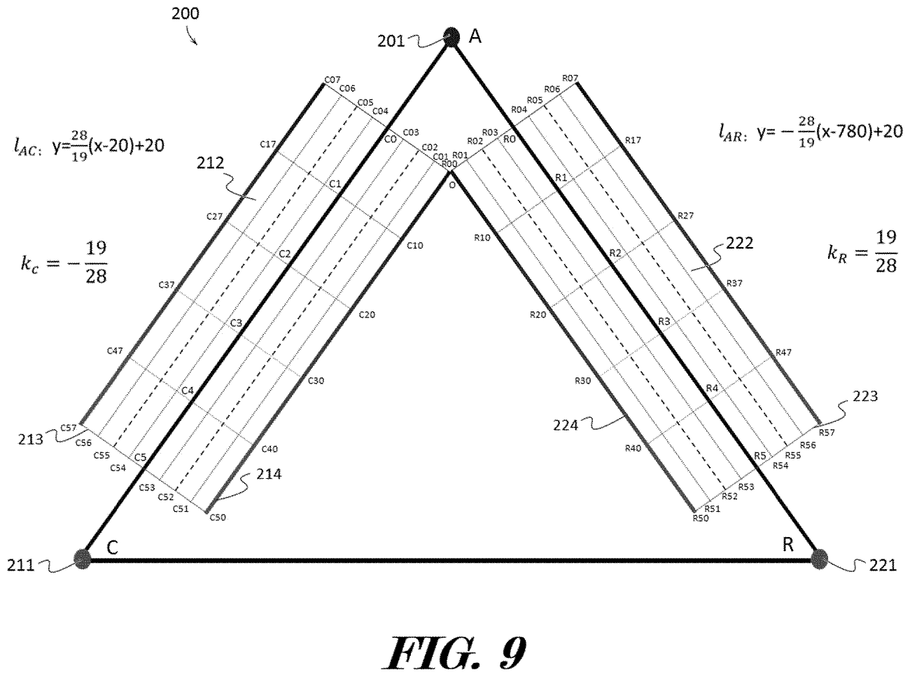

Cancer is a complex disease, and a single treatment therapy is rarely able to cure the disease clinically. Multiple therapies modalities and different treatment schemes are usually required in actual cancer treatment practice. It will require a common universal platform to distinguish information about each treatment response for tracking, evaluation, and sharing with clinicians who have different therapeutic backgrounds. In addition to surgery, the results of systemic therapies and local radiotherapy are associated with the ability of drug distribution and local microcirculation of the tumor. In the present invention, a special infographic has been devised for displaying tumor treatment response information (FIG. 2). The cancer treatment response information diagram (“the diagram”) 200 comprises two symmetric OPP %-Vt % coordinate graph. Two OPP %-Vt % coordinate graph can be mirrored and projected to each other. Left side represents the data of current systemic treatment scheme, right side the data of previous treatment scheme history. If the current treatment is a combination of systemic treatment and radiotherapy, the systemic treatment data is plotted on the left OPP %-Vt % coordinate graph, and the local radiotherapy data is plotted on the right OPP %-Vt % coordinate graph. It can help to monitor and distinguish the effects of different treatment (FIG. 8). Unified standards for infographics have been used to monitor, evaluate, and track treatment responses (FIGS. 5-8). Two breast cancer cases (FIGS. 4A-4C and FIGS. 4D-4F) and their response to chemotherapy are shown in cancer treatment response information diagrams 200 of FIG. 5 and FIG. 6. FIGS. 3A and 3B show examples of a breast cancer tumor 300 and breast 301. In FIGS. 4A-4F, gray area indicates that the area is greater than the threshold, and generating a gray map uses only a single threshold. FIGS. 4A-4C shows an example of an ineffective treatment situation where OPP % is low and the tumor volume reduces small during treatment. FIGS. 4D-4F shows an example of an effective treatment situation where OPP % is high and tumor volume decreases large. According to the change of Vt % following treatment, it will be easily to identify ineffective chemotherapy (FIG. 5) and effective treatment (FIG. 6). Additionally, FIG. 6 may demonstrate a good or positive trend because of high OPP %. In clinic, the high OPP % (better tumor microcirculation) case which shows the better ability of tumor drug distribution may not be associated with the better systemic treatment effects. As shown FIG. 7, a situation that well tumor microcirculation doesn't respond to chemotherapy or targeted therapy and shows an ineffective treatment is demonstrated. Based on the two symmetric coordinate system of infographic, clinicians may review, and analyze the cancer treatment case with chemotherapy combining radiotherapy (FIG. 8). In the cancer treatment response information diagram 200, the tumor's treatment response information point of all measurements during treatment may be dynamically displayed on one cancer treatment response information diagram 200 for evaluating cancer treatment strategy in clinical setting. The detailed design specification of the cancer treatment response information diagram 200 is shown in FIG. 9.

In the present invention, the specially designed information graph and its evaluation criteria can help clinicians including patients to visualize and identify tumor previous response and future possible outcome during treatment. It will be the first time to be able to visualize individual tumor drug resistance causing by pharmacological/physiological factors in the early stage of treatment or even before treatment.

Identifying the Type of Drug Resistance

When cancer treatment is not effective, the clinicians are eager to know the reason of the treatment disorder for optimization of the next treatment plan as early as possible. Medical research has shown that the treatment drug resistance can be divided into two categories: cell-specific factors and pharmacological/physiological factors. Two different types of resistance may require totally different clinical therapeutic strategies to overcome their treatment barriers. At the same time, clinical studies have shown that most human solid tumors exhibit inefficient microcirculation. In other words, most systemic treatment cases may show potential treatment drug resistance causing by low drug distribution/concentration. Although most solid tumors generally have potential drug resistance, systemic therapy has become a common cancer treatment procedure in clinical practice. Effective systemic treatment requires real-time evaluation of drug resistance of each tumor during treatment. How to identify the type of drug resistance is essential for adopting the right treatment strategy to overcome treatment barriers and minimize ineffective treatment or ineffective over-treatment. The emerging precision cancer treatment is a method that provides the most suitable treatment based on the characteristics of each tumor response.

In the present invention, the type of drug resistance can be identified in time by analyzing the cancer treatment response information diagram 200. The identification process may be as follows: If at least two consecutive measurements show that the tumor increases (Vt % is positive value) or the tumor continues to shrink (Vt % is negative value) and their difference is less than the critical value (3%), it means that the tumor occurs treatment drug resistance (FIG. 5 and FIG. 7). This tumor resistance may occur at any stage in the treatment process. When at least two consecutive measurements show that the tumor occurs resistance to treatment and the oxygen perfusion percentage (OPP %) is always lower than 5%, the resistance can be identified as the type of pharmacological/physiological factor (lower drug distribution/concentration and poor microcirculation) (FIG. 5). In this case, clinicians must immediately stop the continuous systemic treatment without waiting for the completion of all systemic treatments. When at least two consecutive measurements show that the tumor occurs resistance to treatment and the oxygenation perfusion percentage (OPP %) are always greater than 20%, it can be identified that the drug resistance is the type of cell-specific factors (FIG. 7). In order to reduce the errors of identification of drug resistance of cell-specific factors, it is necessary to make multiple consecutive measurements. As shown in FIG. 7, at least two treatment response measurements are needed to confirm the type of resistance. In this case, the clinicians can continue the systemic treatment but must immediately change the treatment drugs/agents.

Unlike drug resistance caused by cell-specific factors, drug resistance caused by pharmacological/physiological factors (low drug distribution) can be detected before systemic treatment. This may provide clinical evidence for adjusting the treatment plan to improve the distribution of tumor drugs at the beginning, such as tumor vasculature normalization treatment. It can avoid ineffective systemic therapies (such as, cytotoxic therapy, targeted therapy, etc.) and their side effects. Different from the drug resistance caused by the pharmacological/physiological factors with low drug distribution/concentration, the drug resistance caused by cell-specific factors shows that tumors have good drug distribution capabilities. It provides clinical evidence to continue systemic therapy by switching to the right therapeutic drug/agent.

Herein, considering tumor response to treatment may be delay for a few days, repeating at least two continuous measurements of different time points during treatment is necessary, which can improve the accuracy of identifying the type of drug resistance. It is sufficient for final identification and reduces ineffective treatment (FIGS. 5 and 7). Criteria for identifying drug resistance, such as threshold values for determining OPP % and Vt % of drug resistance types, may depend on analysis of the clinical statistical data. The present invention provides novel technical approaches and methods for identifying types of drug resistance during treatment process.

The uniqueness of the present invention is that it can identify the type of tumor drug resistance in real time during treatment, which can help clinicians to optimize treatment progress and greatly improve the quality of patient's life. Because the drug resistance and its types may dynamically change in response to different treatment schemes, our innovative method will allow to dynamically identify the type of drug resistance and optimize the treatment plan according to the drug resistance type of individual tumors during treatment.

Computerizing the Invention for Clinical Application

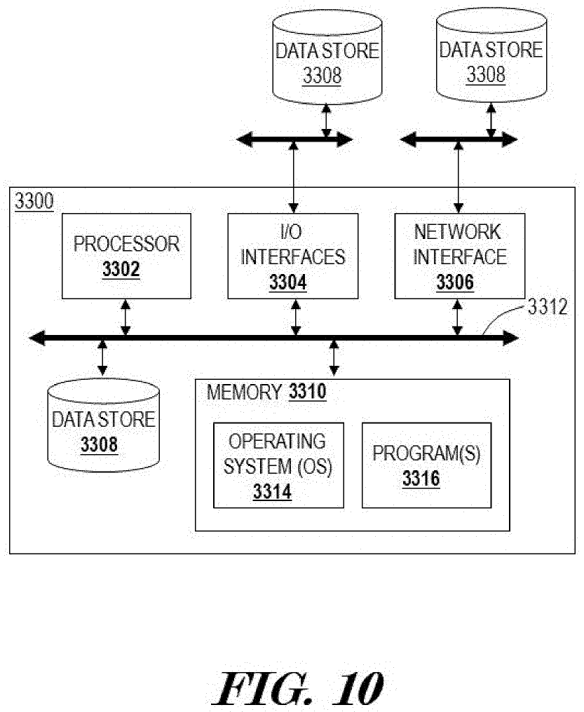

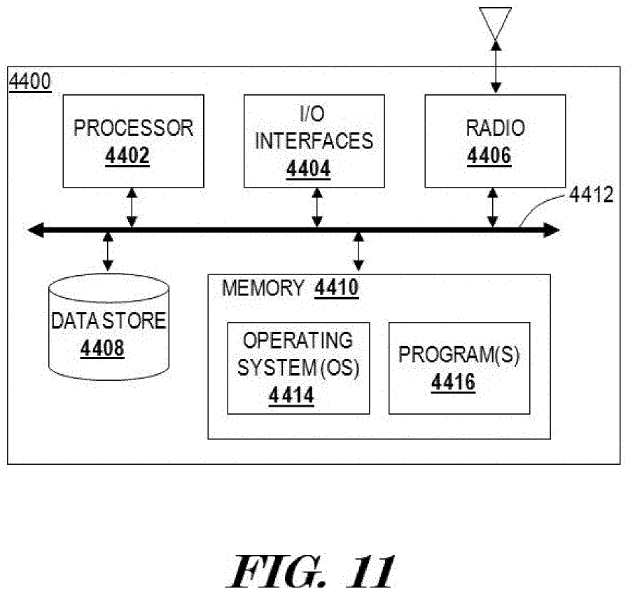

The present invention will now be described and computerized by example, algorithms, and through referencing the appended figures representing preferred and alternative embodiments. FIG. 1 illustrates a block diagram of an example of a computer implemented the identification method (“the method”) 100 according to various embodiments. In some embodiments, one or more steps 110-120 may be performed on an electronic device 4400 (FIG. 11) and/or on a server 3300 (FIG. 10). The method 100 may be used to create a cancer treatment response information diagram 200 (FIGS. 5-8) for treatments including, but not limited to Blood-borne systemic therapies, such as Chemotherapy, Molecularly Targeted therapy, Immunotherapy, Gene therapy, and Photodynamic therapy, Irradiation therapies, such as Radiotherapy, and Combination therapies, such as chemotherapy-radiotherapy, immunotherapy-radiotherapy, molecularly targeted therapy-radiotherapy, radiosensitizer-radiotherapy, other Blood-borne systemic therapies-irradiation therapies for a particular patient 501. In some embodiments, one or more steps 110-121 may be performed before, during, or after a cancer therapy treatment. In further embodiments, one or more steps 110-121 may be performed during, before, or after a cancer therapy treatment scheme. In some embodiments, the method 100 may be used for the treatment of human solid tumors, although in further embodiments, the method 100 may be used for the treatment of solid tumors in any mammal or other organism.

In some embodiments, the method 100 may start 110 and the tumor oxygenated perfusion may be detected by using a Flow and Oxygenation Dependent (FLOOD) MRI (dynamic contrast enhancement T2 weighted MRI) technique, which is sensitive to both variation of oxyhemoglobin concentration and blood flow velocity and flowing direction to MRI static magnetic field B₀. MRI phantom studies have proved that the contribution of MRI signal intensity caused by changes in blood oxyhemoglobin concentration will exceed 10 times the change in blood flow velocity. In further embodiments, the tumor oxygenated perfusion may be detected having the patient breathe air to acquire baseline data in step 111. Next, after inhalation of hyperoxia gas to generate endogenous contrast agent and higher than baseline HbO₂ blood circulating in body, the enhanced data may be acquired in step 112. When higher HbO₂ blood flow through tumor region comparing with difference of HbO₂ between baseline breathing air and hyperoxia gas in same region, the dynamic T2-weighted MRI technique can detect an enhanced MRI signal intensity which is positively related to difference range of HbO₂ in same region. In further embodiments, the pre-treatment MRI measurement may be taken as control and compared with following measurements during the course of treatment. The tumor volume (V₀) of before first treatment can serve as a reference value for calculating volume change ratio during evaluation of the course of treatment. The step 111 and step 112 are from published papers (common knowledge).

In further embodiments, step 111 and/or step 112 may be performed by an Input/Output (I/O) Interface 4404 (FIG. 11), 3304 (FIG. 10), of a server 3300 and/or an electronic device 4400. The data acquired in steps 111 and 112 may be stored in a data store 4408 (FIG. 11), 3308 (FIG. 10), and be accessible to a processor 4402 (FIG. 11), 3302 (FIG. 10). The processor 4402, 3302, may then calculate the region-of-interest (ROI) volume (Vt) of the tumor, which may be performed by volume contour tracing/region-of-interest (ROI) analysis 3D tumor volumetry methods in step 113 which is based on intensity threshold of the T2-weighted Mill images. The tumor regions generally show relatively high signal intensity in T2-weighted Mill images comparing with around normal tissue. The tumor ROI region define and alone gave unacceptable overlap of intensity distributions for tumor and normal tissue. In some cases, it may need to do original data processing for motion correction before analyzing data. The step 113 is from common knowledge.

Next, in step 114, the processor 4402 (FIG. 11), 3302 (FIG. 10) may calculate voxel's enhanced signal intensity (ΔSI). In some embodiments, data analysis may be performed on a voxel-by-voxel basis.

The relative signal intensity (ΔSI) of each tumor voxel may be calculated using the equation:

$$Δ SI = \frac{( S {I}_{E} - S {I}_{b} )}{S {I}_{b}} \% \qquad ( 1 )$$

Where, SI_{E} refers to the enhanced signal intensity in the voxel during inhalation of hyperoxia gas; SI_{b} refers to the average of baseline images in same voxel during inhalation of air. The mean signal intensity-time curve of tumor is used to evaluate quality of measurement. The smooth processing is used to eliminate unstable points due to patient motion. The step 114 is from common knowledge. The data processing algorithm can minimize the influence of blood flow velocity and direction on the MRI enhanced signal.

In step 115, tumor oxygenated perfusion percentage (OPP) may be calculated by the processor 4402 (FIG. 11), 3302 (FIG. 10). The threshold A may be selected as classify high and low contrast enhanced signal (ΔSI) in voxel basis in order to assess whole tumor oxygenated perfusion status. The voxels of the relative signal intensity (ΔSI) being higher than threshold A is counted as high oxygenated perfusion voxel. The percentage of the higher oxygenated perfusion voxel is counted and defined as parameter for evaluating tumor oxygenated perfusion. The higher oxygenated perfusion percentage represents tumor with more oxygenated perfusion inside tumor and better drug/agent/oxygen delivery and distribution. The oxygenated perfusion percentage factor of tumor can be quantified by following equation:

$$( OPP ) \% = \frac{{Σ}_{voxel} ( mean ( Δ {SI}_{voxel} ) > A )}{Total tumor voxel} \% \qquad ( 2 )$$

Where, ΔSI refers to each voxel's relative signal intensity during the patient's hyperoxia gas inhalation; The A refers to the threshold for classifying each voxel as high enhanced relative intensity, which selects as a percentage value based on the MR imaging pulse sequence, TR/TE time, thickness of slices, magnet strength of clinical scanner, sensitivity of coil, cancer site, and etc. For example, it can be assumed a standard threshold 10% for 1.5T and 15% for 3T MRI scanner. The OPP % factor represents the how many percent of tumor regions with oxygenated perfusion above threshold level A, which is an important prognostic factor for next systemic therapy and can be dynamic changed with treatment course. The higher OPP % represents the ability of tumor with the better oxygenated perfusion. Conversely, the lower OPP % represents the lower oxygenated perfusion in tumor region, thereby clarifying the prognostic value of tumor oxygenated blood perfusion.

Next, in step 116, the different threshold set can be processed and different threshold maps may be calculated by processor 4402 (FIG. 11), 3302 (FIG. 10) such as a reconstruction OPP % pseudo color image for better visualization. Several threshold values (such as 0%, 5%, 10%, 20%, and 30%) can be used to classify each voxel and respectively pseudo color value. For example, assign different pseudo color values (1, 50, 100, 150, 200, 250) to voxel's relative signal intensity (ΔSI) respectively (<0%, 0%˜5%, 5%˜10%, 10%˜20%, 20%˜30%, >30%) which respectively correspond to a color table (dark blue, blue, light blue, brown, purple, red). Each voxel of tumor only has one pseudo color value. The tumor pseudo color map data set is completed for display on a display input/output device 3304, 4404. Meanwhile the different threshold values may be used to process in step 115 for calculating different threshold OPP % histogram map for analyzing previous treatment response.

In step 117 the tumor volume change ratio (Vt %) may be calculated by the processor 4402 (FIG. 11), 3302 (FIG. 10). The total tumor volume before first treatment is defined as the reference volume V₀. Each measurement of tumor volume during treatment can be calculated by accounting tumor region Vt.

$$( {V}_{t} ) \% = \frac{( {V}_{t} - {V}_{o} )}{{V}_{o}} \% \qquad ( 3 )$$

Where, V₀ is the tumor original volume before first treatment; Vt is the volume of tumor during treatment. Before first treatment, Vt %=0; if the tumor shrinks, Vt % shows a negative value; if the tumor completely responds to treatment and disappears, Vt %=−100%; if the tumor volume increases during treatment, Vt % shows a positive value. The Vt % parameter directly correlates to cancer response to previous treatment. The step 117 is from common knowledge.

In step 118, special threshold maps may be created by the processor 4402 (FIG. 11), 3302 (FIG. 10) to visualize the data such as using reconstruction OPP % to form pseudo color image of the data in step 116. The three-dimensional pseudo color map data set can be used to visualize tumor different oxygenated perfusion distribution and therapeutic response. 2D pseudo images may displayed as slice by slice on an I/O Interface 4404 (FIG. 11), 3304 (FIG. 10), printer or display screen of a server 3300 and/or an electronic device 4400. The image may be displayed either all pseudo color or only interested pseudo colors image for visualization. For example, by selecting brown, purple, and red color, it may display tumor high oxygenated perfusion area which may be used to evaluate the tumor prognostic information. The dark blue and blue regions correlate to regions of low/non oxygenated perfusion. By selecting dark blue, blue, and light blue colors it may display the tumor low/non oxygenated perfusion image which may be used to monitor the change this part during the course of treatment. As low/non oxygenated perfusion regions of tumor displaying dark blue, blue, and light blue color region of images, they may be fussed to radiation treatment plan for functional image guided irradiation therapy. Next in step 119, the OPP % and Vt % may be drawn on a cancer treatment response information diagram 200 (FIGS. 5-8) which may be displayed on an I/O Interface 4404, 3304, printer or display screen of a server 3300 and/or an electronic device 4400. In some embodiments, a reconstruction tumor oxygenated perfusion percentage OPP % pseudo color image may be displayed or during the course of the cancer treatment on a display input/output device 4404, 3304. In further embodiments, the oxygenated perfusion percentage data OPP % and volume change ratio Vt % obtained before first cancer treatment course may be plotted and the oxygenated perfusion percentage data OPP % and volume change ratio Vt % obtained during the cancer treatment course may be plotted on the cancer treatment response information diagram 200. In still further embodiments, the oxygenated perfusion percentage data OPP % and volume change ratio Vt % data obtained during the current cancer therapy modality for a particular patient 501 may be plotted on the left OPP %-Vt % coordinate graph 212 extending from the poor oxygenated perfusion apex 201 and the first well oxygenated perfusion apex 211 of the cancer treatment response information diagram 200, and wherein the oxygenated perfusion percentage data OPP % and volume change ratio Vt % data obtained from previous therapy modalities history for the particular patient 501 may be plotted on the right OPP %-Vt % coordinate graph 222 extending from the poor oxygenated perfusion apex 201 and the second well oxygenated perfusion apex 221 of the cancer treatment response information diagram 200. At the same time, if the current treatment is a combination of systemic treatment and local radiotherapy, the systemic treatment data is plotted on the left OPP %-Vt % coordinate graph 212, and the radiotherapy data is plotted on the right OPP %-Vt % coordinate graph 222.

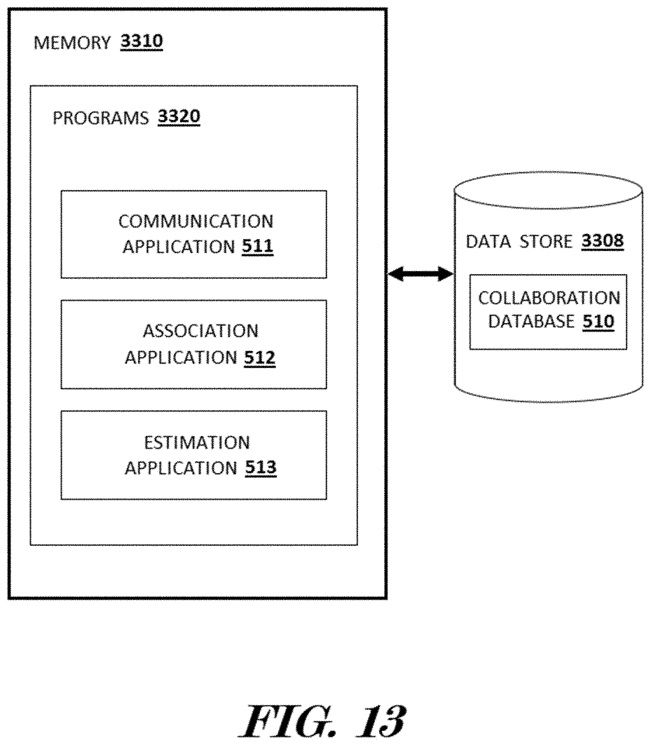

Next in step 120, the type of treatment resistance may be identified by an estimation application 513 (FIG. 13) based on the pooled cancer therapy data of one or more other patients which may be stored in a patients' database 510 (FIG. 13). If continuous measurements showed that the tumor volume increases or the shrinkage value of tumor volume change rate (Vt %) was smaller than 3% and the oxygen perfusion percentage (OPP %) are all less than 5%, it can be identified the treatment resistance caused by low drug distribution in systemic therapies (FIGS. 4A-4C, FIG. 5). The resistance of low drug distribution can be detected and identified before systemic treatments. If continuous measurements showed that the tumor volume increases or the shrinkage value of tumor volume change rate (Vt %) was smaller than 3% and the oxygen perfusion percentage (OPP %) are all higher than 20%, the drug resistance can be identified as the type of cells-specific factors (FIG. 7). Based on the accumulated patients' response data, the critical values for classifying the type of the drug resistance, such as low tumor volume change ratio, low and high oxygenated perfusion percentage (OPP %), may be modified. In further embodiments, the oxygenated perfusion percentage data OPP % and volume change ratio Vt % data for a particular patient 501 may be compared to a database, such as a collaboration database 510, containing a pool of cancer therapy data, oxygenated perfusion percentage data OPP %, and volume change ratio Vt % for one or more other patients 501 to provide an identification of the treatment resistance analysis for a cancer systemic therapy to the particular patient After step 120, the method 100 may end 121.

FIG. 9 provides an example construction of a cancer treatment response information diagram 200 according to various embodiments described herein. It should be understood that a cancer treatment response information diagram 200 may be drawn or composed in any shape, but to further understanding of the invention, some example equations are provided which may be used to construct all or portions of a cancer treatment response information diagram 200. In this example, the diagram 200 may be constructed with an area of 800 by 600 pixels, although other sizes and scales may be used, with the coordinates of A being (400,580), C being (20,20), R being (780,20), ROO being (400,437), COO being (400,437), 0 being (400,437).

In some embodiments, the 201 to 211 side (side AC) of the diagram 200 may be drawn according to the following equation:

l_{AC}: y=28/19(x−20)+20 (4)

In some embodiments, the slope of the 201 to 211 side (side AC) of the diagram 200 may follow the equation:

k_{c}=−19/28 (5)

In some embodiments, the 201 to 221 side (side AR) of the diagram 200 may be drawn according to the following equation:

l_{AR}: y=−28/19(x−780)+20 (6)

In some embodiments, the slope of the 201 to 221 side (side AR) of the diagram 200 may follow the equation:

k_{R}=19/28 (7)

FIG. 2 illustrates an example of a novel cancer treatment response information diagram (“the diagram”) 200 of infographic according to various embodiments described herein. In some embodiments, the diagram 200 may comprise two independent symmetrical coordination systems as a triangle structure comprising three apexes which may be oriented to different cancer therapy modalities. The poor oxygenated perfusion apex 201, optionally oriented at the top of the triangle, may indicate cancer tumors with poor oxygenated perfusion, current systemic therapy-well oxygenated perfusion apex 211, and previous therapeutic modality history or irradiation therapy-well oxygenated perfusion apex 221, optionally oriented at the bottoms of the triangle, may indicate cancer tumors with well oxygenated perfusion. In this non-limiting example, the current systemic therapy-well oxygenated perfusion apex 211 is used to graph blood-borne therapy data, and the previous therapeutic modality history or irradiation therapy-well oxygenated perfusion apex 221 is used to graph irradiation therapy data. In other embodiments, data of any therapy may be graphed on any desired apex or side of the diagram 200. Additionally, a change in tumor volume coordinate graph 212, 222, may extend from both of the two sides, such as the 201 to 211 side and the 201 to 221 side of the diagram 200. In this manner the 201 to 211 side and the 201 to 221 side of the diagram 200 can be used as a coordinate graphing system which each side functioning as a coordinate graphing system for different cancer therapy modality or previous treatment response history. For example, the left coordinate graph 212 of the 201 to 211 side may function as a graphing system for a blood-borne drug/agent therapy and the right coordinate graph 222 of the 201 to 221 side may function as a graphing system for an irradiation therapy or the previous treatment modality history. In further embodiments, the diagram 200 may have any number of sides and each side may represent any therapy.

Preferably, each change in tumor volume coordinate graph 212 and 222 may comprise an oxygenated perfusion percentage (OPP %) x-axis 214 and 224 which may be used to graph oxygenated perfusion percentage (OPP %) data and each change in tumor volume coordinate graph 212, 222, may also comprise a tumor volume change ratio (Vt %) y-axis 213 and 223 which may be used to graph tumor volume change ratio (Vt %) data. In this example, negative values on the tumor volume change ratio (Vt %) y-axes 213 or 223 may be plotted inside the triangular shaped diagram 200, while positive values on the tumor volume change ratio (Vt %) y-axes 213 or 223 may be plotted outside the triangular shaped diagram 200. Also in this example, smaller values on the oxygenated perfusion percentage (OPP %) x-axes 214 or 224 may be plotted closer to the poor oxygenated perfusion apex 201 of the triangular shaped diagram 200, while greater values on the oxygenated perfusion percentage (OPP %) x-axes 214 or 224 may be plotted closer to the first 211 and second 221 therapy-well oxygenated perfusion apexes of the triangular shaped diagram 200.

Each measurement, such as those recorded in steps 115 and 117 of the method 100 (FIG. 1) may result with two values (the cancer oxygenated perfusion percentage (OPP %)) and the volume change ratio (Vt %) which may be expressed as one solid point in the coordinate system. A long axis, such as the 201 to 211 side and the 201 to 221 side, of two coordination graphs between 0%˜100% represents tumor parameter OPP %, which the higher OPP % value correlates higher oxygenated blood perfusion and better drug/agent/oxygen delivery in tumor region and relative high dose distribution and oxygenation level around vessel. The short axis extending from the two sides of coordination graph between −100%˜100% represents the therapeutic response in volume domain, where −100% means a clinical complete response and volume change ratio between −100%˜−30% means tumor shrinkage and shows clinical partial response as shown in FIGS. 5-8, change between −30%˜0% means clinical stable and positive percentage means an increase of tumor volume during treatment. If a cancer has therapeutic complete response, the OPP % of the treatment response information point is marked using previous OPP % value and Vt % is marked −100%. The OPP factor on long axis (201 to 211 side and the 201 to 221 side) represents cancer prognostic information correlating to next outcomes; the volume ratio on short axis (extending from both short axes) represents the cancer response to treatment.

The two separated coordination graphs being mirrored and projected to each other which can be used to evaluate two different treatment modalities. For example, the left side (201 to 211 side) of a triangular diagram 200 may be assigned to evaluate treatment modalities or treatment schemes which are mostly depending on blood-borne system therapeutic molecules, particles, and cells therapies (such as chemotherapy, immunotherapy, gene therapy, photodynamic therapy, and developing molecularly targeted therapy, etc.), while the right side coordination graph (201 to 221 side) of the triangular diagram 200 may be assigned to evaluate local irradiation therapy modalities or previous treatment response history (such as, hyperthermia therapy, radiation therapy, etc.). In some embodiments, the left side coordination graph (201 to 211 side) of a triangular diagram 200 may be assigned to evaluate the current systemic treatment modality or treatment scheme and the right side coordination graph (201 to 221 side) of a triangular diagram 200 may be assigned to evaluate the second cancer treatment modality if the current treatment is a combination cancer treatment. The cancer treatment modality and corresponding treatment response history may include, but is not limited to, chemotherapy, molecular targeted therapy, immunotherapy, gene therapy, photodynamic therapy, radiation therapy, hyperthermia therapy, chemotherapy-radiotherapy combinations, molecular targeted therapy-radiotherapy combinations, immunotherapy-radiotherapy combinations, gene therapy-radiotherapy combinations, photodynamic therapy-radiotherapy combination, radiosensitizer-radiotherapy combination.

Turning now to FIGS. 5-8, oxygenated perfusion percentage data OPP % and volume change ratio Vt % from multiple measurement points, such as during a cancer therapy treatment course or treatment scheme may be plotted on a cancer treatment response information diagram 200

The cancer treatment response information diagrams 200 of two cases (FIGS. 4A-4C, 4D-4F) in chemotherapy are shown in FIGS. 5 and 6. The lower oxygenated perfusion percentage (OPP %) demonstrates lower ability in drug/agent delivery, and lower dose concentration distribution in tumor region and following ineffective treatments (FIG. 5). The higher oxygenated perfusion percentage case correlates more effective drug/agent delivery and higher dose/agent concentration distribution and better outcomes (FIG. 6).

FIG. 7 illustrates an example of a cancer treatment response diagram 200 which describes an ineffective chemotherapy or targeted therapy according to various embodiments described herein. As shown in FIG. 7, although high OPP % values of the two consecutive measurements were taken during treatment, the volume ratio of tumor Vt % only changed a small amount. In this situation, drug resistance can be identified as cell-specific factors. With development of targeted therapy, the mutation of cancer cells may often cause the failure of treatment, which has been reported in professional publications. This case may demonstrate the new method of the present invention to identify the drug resistance of cells-specific factors in clinical routine. It will provide patients and clinicians more time and opportunities to adjust treatment strategy for precision cancer treatment. FIG. 8 shows an example of a cancer treatment response information diagram 200 which describes an effective chemo-radiotherapy combination cancer treatment according to various embodiments described herein. The OPP % value being projected in both asymmetric both coordination systems represent the ongoing chemotherapy or radiotherapy; the tumor volume parameter is marked at each side for evaluating the corresponding therapy results.

Combination cancer therapy as a common treatment modality has been widely used in clinical routine. The systemic treatment plus local irradiation treatment modality (such as, chemotherapy-radiotherapy and immune-radiotherapy etc.) can use both coordination systems on a triangular diagram 200 for tracking and evaluation. For example, the tumor OPP % information can be marked on the long axis of left (the 201 to 211 side) coordination graph, which means an ongoing chemotherapy or immunotherapy. The symmetrical position on the long axis of right (the 201 to 221 side) coordination graph also is projected the same marker of OPP %. Combining tumor volume information Vt %, one solid point is determined and marked on right coordination system which means an ongoing radiotherapy. The diagram 200 of combination therapy can be used to comprehensively analyze the consequence of each treatment modality. It also can be used to evaluate the special monotherapy of radiotherapy combining radio-sensitizer injection. If patient needs a continuing monotherapy, this diagram 200 can continue to draw results on one of coordination systems as previous description of monotherapy.

As an important parameter, the higher OPP % relates to more effective drug/agent/oxygen delivery and oxygenation distribution. Since the result of combination therapy is the comprehensive effect of both treatments, the higher OPP % can be a benefit to both therapy modalities (systemic therapies and local radiation therapy). FIG. 8 demonstrates an ideal case of chemo-radiotherapy for tracking and evaluating during treatment course with a cancer treatment response information diagram 200. It also can be used to evaluate other combination therapies such as immune-radiotherapy, monotherapy such as radiosensitizer-radiotherapy, or any other type of therapy.

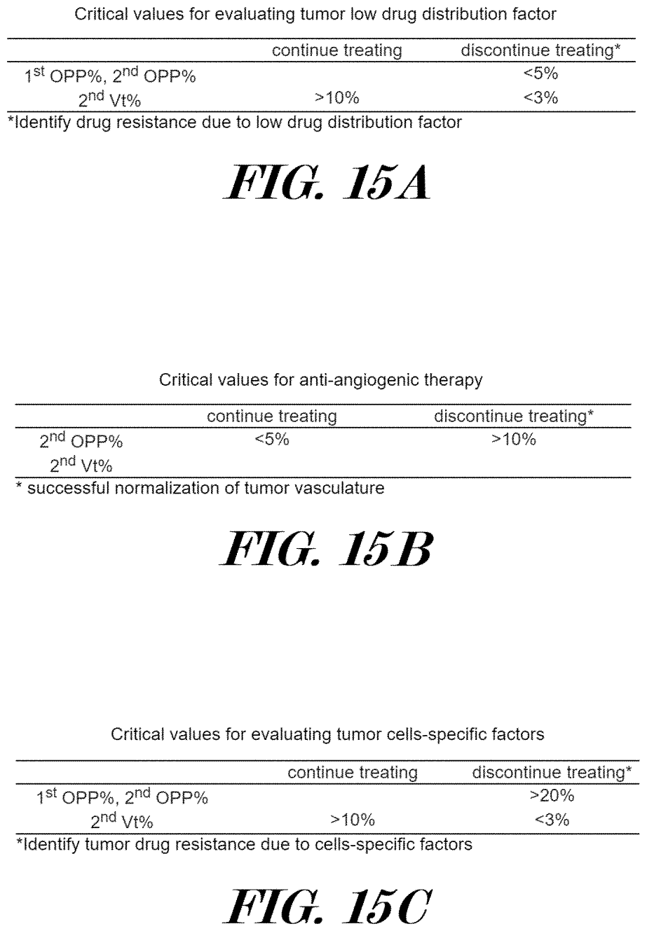

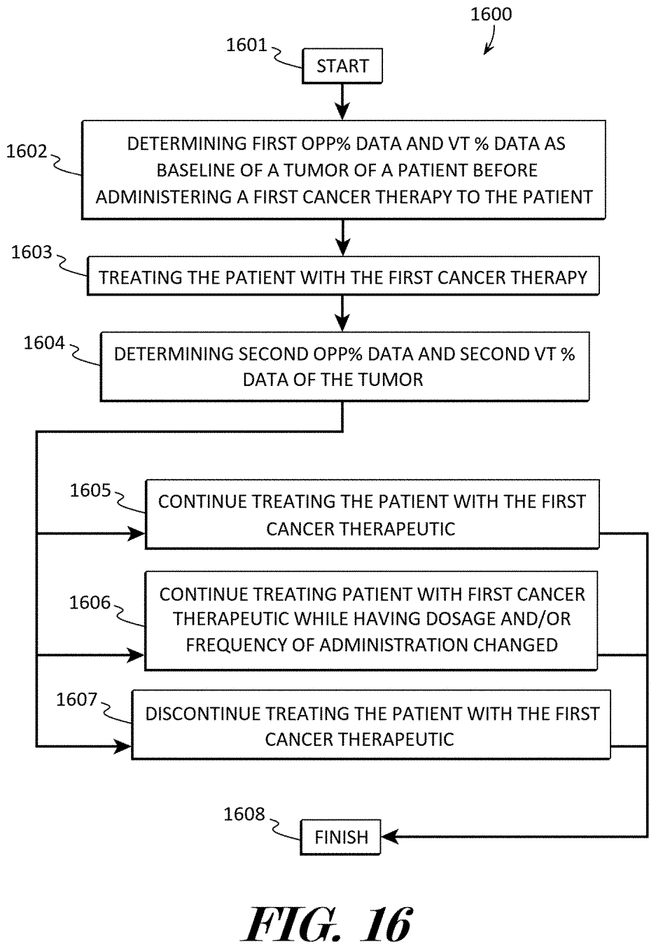

As an example of treatment protocols, and referring to FIGS. 15A-16, anti-angiogenic therapy as one option may be used to treat drug resistance with poor drug distribution. Anti-angiogenic therapies can be used to damage tumor vasculature and alter the hemodynamics and microcirculation inside the tumor, so called normalization of the vasculature for treatment of cancer. However, prescriptions for normalization of the vasculature may depend on the case by case. Individual tumors should have different drugs and treatment dose options because any overdose or overtreatment may lead to opposite results.

Although the treatment and dose are precisely designed by clinicians, the key point of this therapeutic strategy in clinical application is to effectively monitor progress of tumor vascular normalization. In addition to the increase in tumor volume (positive Vt %) showing obviously resistance during treatment, the present invention can be used to accurately monitor tumor response to treatment and optimize the therapeutic plan and systemic prescription doses in real time, which may be the best choice clinically. In other words, it will be of a unique advantage in monitoring tumor vasculature normalization and overcoming tumor drug resistance.

FIG. 16 illustrates a block diagram of a further example method for precision cancer treatment by identifying drug resistance (“the method”) 1600 according to various embodiments described herein. The method 1600 may start 1601 and a first measurement of oxygenated perfusion percentage (OPP %) data and volume change ratio (Vt %) data as baseline of a tumor of a patient before administering the assuming first therapy modality C to the patient may be determined in step 1602.

In step 1603, the patient may be treated with the cancer therapy modality C.

In step 1604, the second measurement of oxygenated perfusion percentage (OPP %) data and a second volume change ratio (Vt %) data of the tumor may be determined.

Next, the method 1600 may proceed to step 1605, step 1606, or step 1607.

In step 1605, the patient may continue to be treated with the cancer therapeutic modality C so that their therapy is unchanged. After step 1605, the method 1600 may finish 1608.

In step 1606, the patient may continue to be treated with the cancer therapeutic modality C while having the dosage and/or frequency of administration changed, such as by being increased or decreased. After step 1606, the method 1600 may finish 1608.

In step 1607, the cancer therapeutic modality C may be discontinued for being administered to the patient. After step 1607, the method 1600 may finish 1608.

### Method 1600 Example 1: Determine the Drug Resistance of Pharmacological/Physiological Factors

In some embodiments, method for precision cancer treatment by identifying drug resistance 1600 may be used to show low drug distribution leading to treatment resistance. In this example, the method 1600 may comprise: determining first measurement of the oxygenated perfusion percentage (OPP %) and a tumor volume (V₀) as reference value of a tumor of a patient before first treatment (step 1602); treating the patient with a cancer therapy modality C (step 1603); determining a second measurement of oxygenated perfusion percentage (OPP %) and volume change ratio (Vt %) of the tumor (step 1604); and performing one of: continue treating the patient with the cancer therapeutic modality C if the second measurement of oxygenated perfusion percentage (OPP %) data is substantially equal to the first oxygenated perfusion percentage (OPP %) data and the second measurement of volume change ratio (Vt %) data shows greater than 10% shrinkage (step 1605); and discontinue treating the patient with the cancer therapeutic modality C if at least two consecutive measurements of oxygenated perfusion percentage (OPP %) are all less than 5% and the difference of the second (and third) measurement in volume change ratio (Vt %) are smaller than 3% shrinkage or volume change ratio (Vt %) data are positive (step 1607). Ineffective treatment is determined to have low drug distribution factor drug resistance, treatment should be stopped immediately and other treatment options should be considered, such as anti-angiogenic therapy to normalize the tumor vasculature.

### Method 1600 Example 2: Monitoring and Controlling Tumor Vasculature Normalization Treatment

If the originally planned cancer treatment regimen was systemic therapy or radiotherapy, the drug resistance of the tumor has been determined to be low drug distribution/concentration. In order to improve tumor microcirculation and drug distribution characteristics, a new treatment method that normalizes the tumor vasculature system through anti-angiogenesis therapy has been introduced in the clinic practice. However, how to effectively control anti-angiogenesis therapy will be the key to normalize tumor vasculature. If the anti-angiogenesis therapy is over-treated, the tumor vascular system is damaged, and the tumor microcirculation may further deteriorate and become unrecoverable. If anti-angiogenic therapy is not adequately treated, treatment may have to be continued. The anti-angiogenics for tumor vascular normalization therapy may also excessively prune tumor vessels in a dose and time-dependent manner, which induces hypoxia inside tumor, improvement of tumor microcirculation or capability of fresh blood flowing through tumor area is only standard for evaluating effectiveness of tumor vasculature normalization. In this situation it may only be necessary to check the change of oxygenated perfusion percentage (OPP %) of the tumor. In some embodiments, a method for precision cancer treatment by identifying drug resistance 1600 may be used to monitor normalization of tumor vasculature. In this example, the method 1600 may comprise the steps of: determining first measurement of oxygenated perfusion percentage (OPP %) and volume change ratio (Vt %) as baseline of a tumor of a patient before anti-angiogenic treatment (step 1602)—optionally this may be done by inheriting last measurement results of oxygenated perfusion percentage (OPP %) data and volume change ratio (Vt %) data of the tumor; treating the patient with a anti-angiogenic therapy course (step 1603); determining second measurement of oxygenated perfusion percentage (OPP %) data and volume change ratio (Vt %) data of the tumor (step 1604); and performing one of: continue treating the patient with the anti-angiogenic treatment if the second measurement of oxygenated perfusion percentage (OPP %) data is still less than 5% (step 1605); adjusting the dosage of the first anti-angiogenic therapy course, such as by increasing or decreasing the dosage (step 1606) if the second measurement of oxygenated perfusion percentage (OPP %) does not change; and discontinue treating the patient with the anti-angiogenic treatment if the second measurement of oxygenated perfusion percentage (OPP %) data is higher than 10% (step 1607). It may be time for the clinicians to consider to continue the original treatment plan: systemic treatment or radiation therapy.

### Method 1600 Example 3: Determine the Drug Resistance of the Cells-Specific Factors

In some embodiments, method for precision cancer treatment by identifying drug resistance 1600 may be used to treat cancer caused by the cells-specific factors. In this example, the method may comprise: determining first measurement of oxygenated perfusion percentage (OPP %) data and volume change ratio (Vt %) data before treatment (step 1602); treating the patient with a cancer systemic treatment C (step 1603); determining second measurement of oxygenated perfusion percentage (OPP %) data and volume change ratio (Vt %) data of the tumor (step 1604); and performing one of: continue treating the patient with the cancer therapeutic modality C if at least two consecutive measurements of volume change ratio (Vt %) data showing shrinkage is greater than 10% (step 1605); and discontinue treating the patient with the cancer therapeutic modality C if at least two consecutive measurements of oxygenated perfusion percentage (OPP %) are greater than 20% and the difference of tumor continues to shrink (Vt %) is smaller than 3% or tumor volume increases (Vt % is positive) (step 1607). Ineffective treatment is determined to be resistant of cell-specific factors, and systemic treatment can be continued but the therapeutic drugs/agents should be replaced immediately. The above three examples demonstrate how to apply the drug resistance identification technology of the present invention to improve current cancer treatments. This technology of identifying drug resistance can be used during the treatment process, which is its unique advantage. It also provides clinical technical support for achieving precision cancer treatment, especially, individualized gene and targeted therapy.

The Cancer Genome Atlas (TCGA) program enables scientists and clinicians to know 10 oncogenic signaling pathways and interpret individuals' genetic codes. Drugs that target these signaling pathways are under development. Currently, there are several drugs that have already been approved by the Food and Drug Administration in the US. These drugs directly target genetic changes in the cells, based on the type, size, and the region of the spread of cancer. The use of drugs to target the changes in the DNA is also known as targeted gene therapy.

However, various factors can cause targeted gene mutations and lead to failed targeted therapies. It is reported that the drug resistance of cells-specific factors still exists in targeted therapies. Also, Cancer Genome Atlas (TCGA) program found that 57% of tumors have at least one potentially actionable alteration in their signaling pathways, which means that treatment targeting the genes of these signaling pathways may potentially fail in targeted therapies. Identifying the resistance of cell-specific factors in time will be the first task of clinical application of precision medicine in cancer treatment. The precision medicine in cancer treatment is expected to become a mainstream medicine in the near future, a part of it is already in practice. In other words, precision medicine is most likely to play a great role in future cancer treatment. The present invention provides the ability to identify the types of drug resistance, which can greatly improve future precision cancer treatment. Similarly, the present invention will also play an important role in improving the efficacy of precision cancer treatment clinically. It will be an indispensable tool for precision medicine in cancer treatment.

The drug resistance identification technology of the present invention can be used to optimize cancer therapeutic strategy during treatment. In addition to the increase in tumor volume (positive Vt %) showing obviously treatment resistance and having to change treatment plan, there are different examples of evidence-based cancer treatment strategies based on analysis of tumor shrinkage response (FIG. 15). The threshold values for determining next therapeutic strategies in FIG. 15 can be modified based on clinical data. With different applications of the present invention, clinicians may have more opportunities to customize cancer treatments for achievement of precision medicine in cancer treatment. This will make cancer treatment more controllable and efficient, and ineffective treatment even ineffective ove treatment will be greatly reduced.

Referring to FIG. 10, in an exemplary embodiment, a block diagram illustrates a server 3300 which may be used in the system 500, in other systems, or standalone. The server 3300 may be a digital computer that, in terms of hardware architecture, generally includes a processor 3302, input/output (I/O) interfaces 3304, a network interface 3306, a data store 3308, and memory 3310. It should be appreciated by those of ordinary skill in the art that FIG. 11 depicts the server 3300 in an oversimplified manner, and a practical embodiment may include additional components and suitably configured processing logic to support known or conventional operating features that are not described in detail herein. The components (3302, 3304, 3306, 3308, and 3310) are communicatively coupled via a local interface 3312. The local interface 3312 may be, for example but not limited to, one or more buses or other wired or wireless connections, as is known in the art. The local interface 3312 may have additional elements, which are omitted for simplicity, such as controllers, buffers (caches), drivers, repeaters, and receivers, among many others, to enable communications. Further, the local interface 3312 may include address, control, and/or data connections to enable appropriate communications among the aforementioned components.

The processor 3302 is a hardware device for executing software instructions. The processor 3302 may be any custom made or commercially available processor, a central processing unit (CPU), an auxiliary processor among several processors associated with the server 3300, a semiconductor-based microprocessor (in the form of a microchip or chip set), or generally any device for executing software instructions. When the server 3300 is in operation, the processor 3302 is configured to execute software stored within the memory 3310, to communicate data to and from the memory 3310, and to generally control operations of the server 3300 pursuant to the software instructions. The I/O interfaces 3304 may be used to receive user input from and/or for providing system output to one or more devices or components. User input may be provided via, for example, a keyboard, touch pad, and/or a mouse. System output may be provided via a display device and a printer (not shown). I/O interfaces 3304 may include, for example, a serial port, a parallel port, a small computer system interface (SCSI), a serial ATA (SATA), a fibre channel, Infiniband, iSCSI, a PCI Express interface (PCI-x), an infrared (IR) interface, a radio frequency (RF) interface, and/or a universal serial bus (USB) interface.

The network interface 3306 may be used to enable the server 3300 to communicate on a network, such as the Internet, a wide area network (WAN), a local area network (LAN), and the like, etc. The network interface 3306 may include, for example, an Ethernet card or adapter (e.g., 10BaseT, Fast Ethernet, Gigabit Ethernet, 10 GbE) or a wireless local area network (WLAN) card or adapter (e.g., 802.11a/b/g/n). The network interface 3306 may include address, control, and/or data connections to enable appropriate communications on the network. A data store 3308 may be used to store data. The data store 3308 may include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 3308 may incorporate electronic, magnetic, optical, and/or other types of storage media. In one example, the data store 3308 may be located internal to the server 3300 such as, for example, an internal hard drive connected to the local interface 3312 in the server 3300. Additionally, in another embodiment, the data store 3308 may be located external to the server 3300 such as, for example, an external hard drive connected to the I/O interfaces 3304 (e.g., SCSI or USB connection). In a further embodiment, the data store 3308 may be connected to the server 3300 through a network, such as, for example, a network attached file server.

The memory 3310 may include any of volatile memory elements (e.g., random access memory (RAM, such as DRAM, SRAM, SDRAM, etc.)), nonvolatile memory elements (e.g., ROM, hard drive, tape, CDROM, etc.), and combinations thereof. Moreover, the memory 3310 may incorporate electronic, magnetic, optical, and/or other types of storage media. Note that the memory 3310 may have a distributed architecture, where various components are situated remotely from one another, but can be accessed by the processor 3302. The software in memory 3310 may include one or more software programs, each of which includes an ordered listing of executable instructions for implementing logical functions. The software in the memory 3310 includes a suitable operating system (O/S) 3314 and one or more programs 3316. The operating system 3314 essentially controls the execution of other computer programs, such as the one or more programs 3316, and provides scheduling, input-output control, file and data management, memory management, and communication control and related services. The one or more programs 3316 may be configured to implement the various processes, algorithms, methods, techniques, etc. described herein.

Referring to FIG. 11, in an exemplary embodiment, a block diagram illustrates an electronic device 4400, which may be used in the system 500 or the like. The term “electronic device” as used herein is a type of electronic device comprising circuitry and configured to generally perform functions such as recording audio, photos, and videos; displaying or reproducing audio, photos, and videos; storing, retrieving, or manipulation of electronic data; providing electrical communications and network connectivity; or any other similar function. Non-limiting examples of electronic devices include; personal computers (PCs), workstations, laptops, tablet PCs including the iPad, cell phones including iOS phones made by Apple Inc., Android OS phones, Microsoft OS phones, Blackberry phones, digital music players, or any electronic device capable of running computer software and displaying information to a user, memory cards, other memory storage devices, digital cameras, external battery packs, external charging devices, and the like. Certain types of electronic devices which are portable and easily carried by a person from one location to another may sometimes be referred to as a “portable electronic device” or “portable device”. Some non-limiting examples of portable devices include; cell phones, smart phones, tablet computers, laptop computers, wearable computers such as watches, Google Glasses, etc. and the like.

The electronic device 4400 can be a digital device that, in terms of hardware architecture, generally includes a processor 4402, input/output (I/O) interfaces 4404, a radio 4406, a data store 4408, and memory 4410. It should be appreciated by those of ordinary skill in the art that FIG. 11 depicts the electronic device 4400 in an oversimplified manner, and a practical embodiment may include additional components and suitably configured processing logic to support known or conventional operating features that are not described in detail herein. The components (4402, 4404, 4406, 4408, and 4410) are communicatively coupled via a local interface 4412. The local interface 4412 can be, for example but not limited to, one or more buses or other wired or wireless connections, as is known in the art. The local interface 4412 can have additional elements, which are omitted for simplicity, such as controllers, buffers (caches), drivers, repeaters, and receivers, among many others, to enable communications. Further, the local interface 4412 may include address, control, and/or data connections to enable appropriate communications among the aforementioned components.

The processor 4402 is a hardware device for executing software instructions. The processor 4402 can be any custom made or commercially available processor, a central processing unit (CPU), an auxiliary processor among several processors associated with the electronic device 4400, a semiconductor-based microprocessor (in the form of a microchip or chip set), or generally any device for executing software instructions. When the electronic device 4400 is in operation, the processor 4402 is configured to execute software stored within the memory 4410, to communicate data to and from the memory 4410, and to generally control operations of the electronic device 4400 pursuant to the software instructions. In an exemplary embodiment, the processor 4402 may include a mobile optimized processor such as optimized for power consumption and mobile applications. The I/O interfaces 4404 can be used to receive user input from and/or for providing system output. User input can be provided via, for example, a keypad, a touch screen, a scroll ball, a scroll bar, buttons, bar code scanner, and the like. System output can be provided via a display device such as a liquid crystal display (LCD), touch screen, and the like. The I/O interfaces 4404 can also include, for example, a serial port, a parallel port, a small computer system interface (SCSI), an infrared (IR) interface, a radio frequency (RF) interface, a universal serial bus (USB) interface, and the like. The I/O interfaces 4404 can include a graphical user interface (GUI) that enables a user to interact with the electronic device 4400. Additionally, the I/O interfaces 4404 may further include an imaging device, i.e. camera, video camera, etc.

The radio 4406 enables wireless communication to an external access device or network. Any number of suitable wireless data communication protocols, techniques, or methodologies can be supported by the radio 4406, including, without limitation: RF; IrDA (infrared); Bluetooth; ZigBee (and other variants of the IEEE 802.15 protocol); IEEE 802.11 (any variation); IEEE 802.16 (WiMAX or any other variation); Direct Sequence Spread Spectrum; Frequency Hopping Spread Spectrum; Long Term Evolution (LTE); cellular/wireless/cordless telecommunication protocols (e.g. 3G/4G, or developing 5G etc.); wireless home network communication protocols; paging network protocols; magnetic induction; satellite data communication protocols; wireless hospital or health care facility network protocols such as those operating in the WMTS bands; GPRS; proprietary wireless data communication protocols such as variants of Wireless USB; and any other protocols for wireless communication. The data store 4408 may be used to store data. The data store 4408 may include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 4408 may incorporate electronic, magnetic, optical, and/or other types of storage media.

The memory 4410 may include any of volatile memory elements (e.g., random access memory (RAM, such as DRAM, SRAM, SDRAM, etc.)), nonvolatile memory elements (e.g., ROM, hard drive, etc.), and combinations thereof. Moreover, the memory 4410 may incorporate electronic, magnetic, optical, and/or other types of storage media. Note that the memory 4410 may have a distributed architecture, where various components are situated remotely from one another, but can be accessed by the processor 4402. The software in memory 4410 can include one or more software programs, each of which includes an ordered listing of executable instructions for implementing logical functions. In the example of FIG. 8, the software in the memory 4410 includes a suitable operating system (O/S) 4414 and programs 4416. The operating system 4414 essentially controls the execution of other computer programs, and provides scheduling, input-output control, file and data management, memory management, and communication control and related services. The programs 4416 may include various applications, add-ons, etc. configured to provide end user functionality with the electronic device 4400. For example, exemplary programs 4416 may include, but not limited to, a web browser, social networking applications, streaming media applications, games, mapping and location applications, electronic mail applications, financial applications, and the like. In a typical example, the end user typically uses one or more of the programs 4416 along with a network.

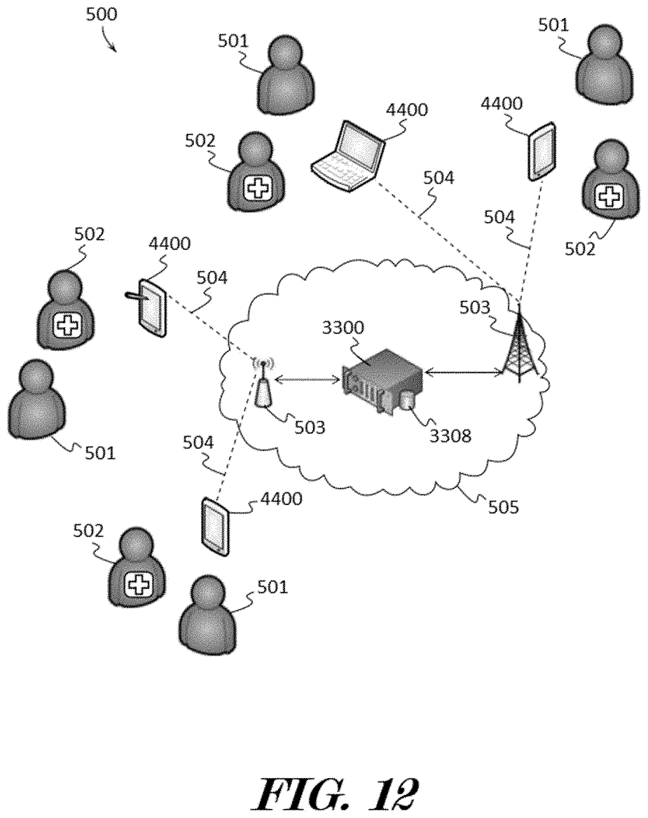

As perhaps best shown by FIG. 12, in some embodiments, as a Therapy-Oriented evaluation tool, a cancer treatment response information diagram 200 can be used as a general platform to share tumor prognostic information between clinicians 502 with different treatment modalities backgrounds to allow for clinician 502 collaboration in optimizing a therapeutic strategy before or during a cancer therapy course of treatment for their patients 501. This collaboration may be performed using a cancer therapy treatment resistance identification system (“the system”) 500. The system 500 may receive the health information of a patient 501, such as one or more diagrams 200, data from one or more diagrams 200, and/or any other data and information related to treatment data, such as sex, age, histopathology, and disease stage, genomic data, treatment plan, which may be stored in a collaboration database 510 and preferably sorted according to treatment site, stage, sex, treatment modality, or any other filtering criteria. Each patient measurement point during a treatment course of a cancer therapy may be collected as therapy response data no matter how effective or ineffective the treatment or therapy is. Based on accumulated and analyzed response data, the system 500 may provide clinicians 502 and patients 501 a quantitative successful probability being calculated by collected similar patient treatment and response data pool and identifying the type of drug resistance for each treatment modality and scheme in order to optimize the therapeutic strategy and achieve precision cancer treatment. In some embodiments, an identification method may include a comparison between treatment effectiveness and patient's quality of life; the possible outcome and side effects and the dose strength of a cancer therapy. In other embodiments, an identification method may include a comparison between the typical rate of tumor response and one or more selected cancer therapies and/or cancer therapy treatment schemes.

An illustrative example of some of the physical components which may comprise a cancer treatment response collaboration system 500 according to some embodiments is presented in FIG. 12. The system 500 is configured to facilitate the transfer of data and information between one or more access points 503, electronic devices 4400, and servers 3300 over a data network 505. Each electronic device 4400 may send data to and receive data from the data network 505 through a network connection 504 with an access point 503. A data store 3308 accessible by the server 3300 may contain one or more databases. The data may comprise any information pertinent to one or more patients 501, clinicians 502, and/or other users which may be input into the system 500 including information on or describing cancer therapy data of one or more patients 501, information requested by one or more clinicians 502, information supplied by one or more clinicians 502, and any other information which a clinician 502 may use for cancer treatment evaluation and collaboration of one or more patients 501.

In this example, the system 500 comprises at least one electronic device 4400 (but preferably more than two electronic devices 4400) configured to be operated by one or more clinicians 502. In some embodiments, the system 500 may be configured to facilitate the communication of information between one or more clinicians 502, through their respective electronic devices 4400 and/or servers 3300 of the system 500. Electronic devices 4400 can be mobile devices, such as laptops, tablet computers, personal digital assistants, smart phones, and the like, that are equipped with a wired or wireless network interface capable of sending data to one or more servers 3300 with access to one or more data stores 3308 over a network 505 such as a wired local area network or wireless local area network. Additionally, user electronic devices 4400 can be fixed devices, such as desktops, imagining devices, medical workstations, treatment and administration workstations, and the like, that are equipped with a wireless or wired network interface capable of sending data to one or more servers 3300 with access to one or more data stores 3308 over a wireless or wired local area network 505. The present invention may be implemented on at least one electronic device 4400 and/or server 3300 programmed to perform one or more of the steps described herein. In some embodiments, more than one user electronic device 4400 and/or server 3300 may be used, with each being programmed to carry out one or more steps of a method or process described herein.

Referring now to FIG. 13, a block diagram showing some software rules engines which may be found in a system 500 (FIG. 12) which may optionally be configured to run on a server 3300 (FIGS. 10 and 12) and an example of a collaboration database 510 according to various embodiments described herein are illustrated, respectively. In some embodiments, one or more servers 3300 may be configured to run one or more software rules engines or programs such as a communication application 511, association application 512, and/or an estimation application 513. In this embodiment, the applications 511, 512, 513, are configured to run on at least one server 3300. The server 3300 may be in electronic communication with a data store 3308 comprising a database, such as a collaboration database 510. The engines 511, 512, 513, may read, write, or otherwise access data in one or more databases of the data store 308. Additionally, data may be sent and received to and from one or more electronic devices 4400 (FIGS. 11 and 12) which may be in wired and/or wireless electronic communication with the server 3300 through a network 505. In other embodiments, a communication application 511, association application 512, and/or an estimation application 513 may be configured to run on an electronic device 4400 and/or server 3300 with data transferred to and from one or more servers 3300 in communication with a data store 3308 through a network 505. In still further embodiments, a server 3300 or an electronic device 4400 may be configured to run a communication application 511, association application 512, and/or an estimation application 513.

In some embodiments, the system 500 may comprise a database, such as a collaboration database 510, optionally stored on a data store 3308 accessible to a communication application 511, association application 512, and/or an estimation application 513. In further embodiments, a collaboration database 510 may be stored on a data store 4408 of an electronic device 4400. A collaboration database 510 may comprise any data and information pertinent to one or more patients 501 and/or clinicians 502 of the system 500. This data may include information which may describe the cancer therapy, results of cancer therapy, and other health information which may describe a patient 501. For example, this health information may include oxygenated perfusion percentage data OPP %, volume change ratio Vt % data, imaging data, types of cancer therapies received, durations of cancer therapies received, doses of cancer therapies received, or any other health information which may describe one or more patients 501 of a clinician 502. Additionally, the data of two or more patients 501 and/or clinicians 502 may be pooled so that the all the information which may describe the cancer therapy, results of cancer therapy, and other health information of all of the patients 501 in the collaboration database 510 may be searched.

The communication application 511 may comprise a computer program which may be executed by a computing device processor, such as a processor 3302 (FIG. 10) and/or a processor 4402 (FIG. 11), and which may be configured to govern electronic communication between severs 3300 and electronic devices 4400. Data from severs 3300 and electronic devices 4400 may be received by the communication application 511 which may then electronically communicate the data to the association application 512 and estimation application 513. Likewise, data from the association application 512 and estimation application 513 may be received by the communication application 511 which may then electronically communicate the data to servers 3300 and electronic devices 4400. In some embodiments, the communication application 511 may govern the electronic communication by initiating, maintaining, reestablishing, and terminating electronic communication between one or more electronic devices 4400 and servers 3300. In further embodiments, the communication application 511 may control the network interface 3306 (FIG. 10) of the server 3300 to send and receive data to and from one or more electronic devices 4400 and other servers 3300 through a network connection 504 (FIG. 12) over a network 505 (FIG. 12).

The association application 512 may comprise a computer program which may be executed by a computing device processor, such as a processor 3302 (FIG. 10) and/or a processor 4402 (FIG. 11), and which may be configured to store, retrieve, modify, create, and/or delete data and information which may describe the cancer therapy, results of cancer therapy, and other health information of a patient 501, including oxygenated perfusion percentage data OPP %, volume change ratio Vt % data, imaging data, types of cancer therapies received, durations of cancer therapies received, doses of cancer therapies received, or any other health information which may describe one or more patients 501 of a clinician 502 into and from the collaboration database 510. In some embodiments, the association application 512 receive data from the communication application 511 and/or estimation application 513 and associate the data with information which may describe the cancer therapy, results of cancer therapy, and other health information of a patient 501, including oxygenated perfusion percentage data OPP %, volume change ratio Vt % data, imaging data, types of cancer therapies received, durations of cancer therapies received, doses of cancer therapies received, or any other health information which may describe one or more patients 501 of a clinician 502 into the collaboration database 510. In further embodiments, the association application 512 retrieve data from the collaboration database 510, such as information which may describe the cancer therapy, results of cancer therapy, and other health information of a patient 501, including oxygenated perfusion percentage data OPP %, volume change ratio Vt % data, imaging data, types of cancer therapies received, durations of cancer therapies received, doses of cancer therapies received, or any other health information which may describe one or more patients 501 of a clinician 502, and send or communicate the data to the communication application 511 and/or estimation application 513.

The estimation application 513 may comprise a computer program which may be executed by a computing device processor, such as a processor 3302 (FIG. 10) and/or a processor 4402 (FIG. 11), and which may be configured to compare data received from the communication application 511 to data received from the association application 512. In some embodiments, the estimation application 513 may compare the health information of a particular patient 501 received by the communication application 511 through the electronic device 4400 of a clinician 502 to the health information of one or more patients 501, including the pooled health information and data of all the patients 501 in the collaboration database 510, retrieved by the association application 512 from the collaboration database 510. The estimation application 513 may be configured to identify the type of drug resistance of how the cancer tumor of the particular patient 501 would respond to a cancer therapy that the particular patient 501 has not yet received based upon the oxygenated perfusion percentage data OPP % and volume change ratio Vt % pooled data of the identified one or patients in the collaboration database 510 that did undergo the cancer therapy that the particular patient has not yet received. Based on the pooled and analyzed response data, the estimation application 513 of the system 500 may provide clinicians 502 and patients 501 a quantitative identification method for each treatment modality and scheme in order to optimize the therapeutic strategy and achieve precision cancer treatment.

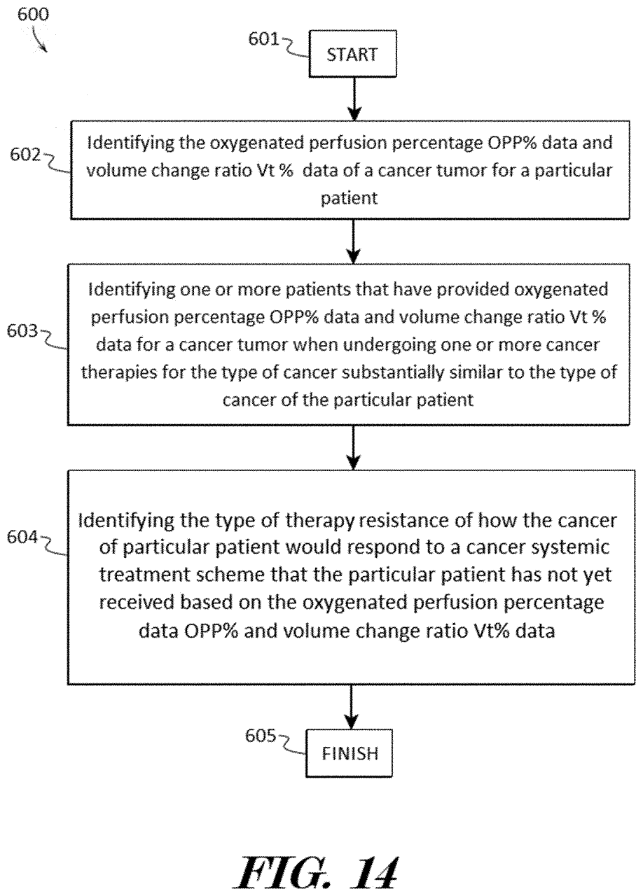

FIG. 14 shows a block diagram of an example of a computer-implemented method for precision cancer treatment by identifying drug resistance (“the method”) 600 which may utilize one or more cancer treatment response information diagrams 200 and a cancer therapy treatment resistance identification system 500 according to various embodiments described herein. In some embodiments, the method 600 may be used to provide clinicians 502 and patients 501 a quantitative identification method for each cancer therapy treatment modality or treatment scheme in order to optimize the therapeutic strategy and achieve precision cancer treatment using one or more electronic devices 4400 and/or servers 3300. One or more steps of the method 600 may be performed by a communication application 511, an association application 512, and/or an estimation application 513 which may be executed by the processor of an electronic device, such as a processor 3302 (FIG. 10) and/or a processor 4402 (FIG. 11). In some embodiments, the method 600 may be used for the treatment of human solid tumors, although in further embodiments, the method 600 may be used for the treatment of solid tumors in any mammal or other organism.

In some embodiments, the method 600 may start 601 and the oxygenated perfusion percentage data OPP % and volume change ratio Vt % data of a cancer tumor for a particular patient 501 (FIG. 12) may be identified in step 602. In further embodiments, step 602 may be performed using steps 110-115 of the cancer drug resistance identification method 100 of FIG. 1. In still further embodiments, step 118 and/or 119 of the cancer drug resistance identification method 100 of FIG. 1 may also be performed in step 602. This data may be communicated by a communication application 511 (FIG. 13) and an association application 512 (FIG. 13) to a collaboration database 510 (FIG. 13).

Next, in step 603 one or more patients 501 that have provided oxygenated perfusion percentage data OPP % and volume change ratio Vt % data, such as by one or more steps of the cancer drug resistance identification method 100 of FIG. 1, for a cancer tumor when undergoing one or more cancer therapies for the type of cancer substantially similar to the type of cancer of the particular patient 501 may be identified in the collaboration database 510 by the association application 512. Preferably, the association application 512 may retrieve this data without retrieving any personally identifying information of the one or more patients 501.

In step 604, an identification method of how the cancer tumor of the particular patient 501 would respond to a cancer therapy treatment scheme that the particular patient 501 has not yet received may be generated by the estimation application 513 based upon the oxygenated perfusion percentage data OPP % and volume change ratio Vt % pooled data in the collaboration database 510 of the identified one or patients 501 that did undergo the cancer therapy treatment scheme that the particular patient 501 has not yet received. In some embodiments, the identification method may include how the percentage of tumor complete response and partial response for each particular therapeutic modality. In further embodiments, an identification method may include a comparison between treatment effectiveness and patient's quality of life; the possible outcome and the side effects and the dose strength of a cancer therapy. In other embodiments, an identification method may include a comparison between the typical rate of tumor response and one or more selected cancer therapies and/or cancer therapy treatment schemes. An identification method may be generated for each cancer therapy that has been administered to one or more patients having health information, such as oxygenated perfusion percentage data OPP % and volume change ratio Vt % data, for a substantially similar type of cancer as the particular patient 501. After step 604, the method 600 may finish 605.

It will be appreciated that some exemplary embodiments described herein may include one or more generic or specialized processors (or “processing devices”) such as microprocessors, digital signal processors, customized processors and field programmable gate arrays (FPGAs) and unique stored program instructions (including both software and firmware) that control the one or more processors to implement, in conjunction with certain non-processor circuits, some, most, or all of the functions of the methods and/or systems described herein. Alternatively, some or all functions may be implemented by a state machine that has no stored program instructions, or in one or more application specific integrated circuits (ΔSICs), in which each function or some combinations of certain of the functions are implemented as custom logic. Of course, a combination of the two approaches may be used. Moreover, some exemplary embodiments may be implemented as a computer-readable storage medium having computer readable code stored thereon for programming a computer, server, appliance, device, etc. each of which may include a processor to perform methods as described and claimed herein. Examples of such computer-readable storage mediums include, but are not limited to, a hard disk, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory), a Flash memory, and the like.

Embodiments of the subject matter and the functional operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer program products, i.e., one or more modules of computer program instructions encoded on a tangible program carrier for execution by, or to control the operation of, data processing apparatus. The tangible program carrier can be a propagated signal or a computer readable medium. The propagated signal is an artificially generated signal, e.g., a machine generated electrical, optical, or electromagnetic signal that is generated to encode information for transmission to suitable receiver apparatus for execution by a computer. The computer readable medium can be a machine-readable storage device, a machine-readable storage substrate, a memory device, a composition of matter effecting a machine readable propagated signal, or a combination of one or more of them.

A computer program (also known as a program, software, software application, application, script, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Additionally, the logic flows and structure block diagrams described in this patent document, which describe particular methods and/or corresponding acts in support of steps and corresponding functions in support of disclosed structural means, may also be utilized to implement corresponding software structures and algorithms, and equivalents thereof. The processes and logic flows described in this specification can be performed by one or more programmable processors (computing device processors) executing one or more computer applications or programs to perform functions by operating on input data and generating output.

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, solid state drives, or optical disks. However, a computer need not have such devices.

Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described is this specification, or any combination of one or more such back end, middleware, or front end components. The components of the system can be interconnected by any form or medium of digital data communication, e.g., a communication network such as PACS system. Examples of communication networks include a local area network (“LAN”) and a wide area network (“WAN”), e.g., the Internet.

The computing system can include clients and servers. A client and server are generally remote from each other and typically interact through a communication network or the cloud. The relationship of client and server arises by virtue of computer programs running on the respective computers and having a client server relationship to each other.

Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, this sequence of actions described herein can be considered to be embodied entirely within any form of computer readable storage medium having stored therein a corresponding set of computer instructions that upon execution would cause an associated processor to perform the functionality described herein. Thus, the various aspects of the invention may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

The computer system may also include a main memory, such as a random access memory (RAM) or other dynamic storage device (e.g., dynamic RAM (DRAM), static RAM (SRAM), and synchronous DRAM (SDRAM)), coupled to the bus for storing information and instructions to be executed by processor. In addition, the main memory may be used for storing temporary variables or other intermediate information during the execution of instructions by the processor. The computer system may further include a read only memory (ROM) or other static storage device (e.g., programmable ROM (PROM), erasable PROM (EPROM), and electrically erasable PROM (EEPROM)) coupled to the bus for storing static information and instructions for the processor.

The computer system may also include a disk controller coupled to the bus to control one or more storage devices for storing information and instructions, such as a magnetic hard disk, and a removable media drive (e.g., floppy disk drive, read-only compact disc drive, read/write compact disc drive, compact disc jukebox, tape drive, and removable magneto-optical drive). The storage devices may be added to the computer system using an appropriate device interface (e.g., small computer system interface (SCSI), integrated device electronics (IDE), enhanced-IDE (E-IDE), direct memory access (DMA), or ultra-DMA).

The computer system may also include special purpose logic devices (e.g., application specific integrated circuits (ΔSICs)) or configurable logic devices (e.g., simple programmable logic devices (SPLDs), complex programmable logic devices (CPLDs), and field programmable gate arrays (FPGAs)).

The computer system may also include a display controller coupled to the bus to control a display, such as a cathode ray tube (CRT), liquid crystal display (LCD) or any other type of display, for displaying information to a computer user. The computer system may also include input devices, such as a keyboard and a pointing device, for interacting with a computer user and providing information to the processor. Additionally, a touch screen could be employed in conjunction with display. The pointing device, for example, may be a mouse, a trackball, or a pointing stick for communicating direction information and command selections to the processor and for controlling cursor movement on the display. In addition, a printer may provide printed listings of data stored and/or generated by the computer system.

The computer system performs a portion or all of the processing steps of the invention in response to the processor executing one or more sequences of one or more instructions contained in a memory, such as the main memory. Such instructions may be read into the main memory from another computer readable medium, such as a hard disk or a removable media drive. One or more processors in a multi-processing arrangement may also be employed to execute the sequences of instructions contained in main memory. In alternative embodiments, hard-wired circuitry may be used in place of or in combination with software instructions. Thus, embodiments are not limited to any specific combination of hardware circuitry and software.

As stated above, the computer system includes at least one computer readable medium or memory for holding instructions programmed according to the teachings of the invention and for containing data structures, tables, records, or other data described herein. Examples of computer readable media are compact discs, hard disks, floppy disks, tape, magneto-optical disks, PROMs (EPROM, EEPROM, flash EPROM), DRAM, SRAM, SDRAM, or any other magnetic medium, compact discs (e.g., CD-ROM), or any other optical medium, punch cards, paper tape, or other physical medium with patterns of holes, a carrier wave (described below), or any other medium from which a computer can read.

Stored on any one or on a combination of computer readable media, the present invention includes software for controlling the computer system, for driving a device or devices for implementing the invention, and for enabling the computer system to interact with a human user. Such software may include, but is not limited to, device drivers, operating systems, development tools, and applications software. Such computer readable media further includes the computer program product of the present invention for performing all or a portion (if processing is distributed) of the processing performed in implementing the invention.

The computer code or software code of the present invention may be any interpretable or executable code mechanism, including but not limited to scripts, interpretable programs, dynamic link libraries (DLLs), Java classes, and complete executable programs. Moreover, parts of the processing of the present invention may be distributed for better performance, reliability, and/or cost.

Various forms of computer readable media may be involved in carrying out one or more sequences of one or more instructions to processor for execution. For example, the instructions may initially be carried on a magnetic disk of a remote computer. The remote computer can load the instructions for implementing all or a portion of the present invention remotely into a dynamic memory and send the instructions over the air (e.g. through a wireless cellular network or WiFi network). A modem local to the computer system may receive the data over the air and use an infrared transmitter to convert the data to an infrared signal. An infrared detector coupled to the bus can receive the data carried in the infrared signal and place the data on the bus. The bus carries the data to the main memory, from which the processor retrieves and executes the instructions. The instructions received by the main memory may optionally be stored on storage device either before or after execution by processor.

The computer system also includes a communication interface coupled to the bus. The communication interface provides a two-way data communication coupling to a network link that is connected to, for example, a local area network (LAN), or to another communications network such as the Internet. For example, the communication interface may be a network interface card to attach to any packet switched LAN. As another example, the communication interface may be an asymmetrical digital subscriber line (ADSL) card, an integrated services digital network (ISDN) card or a modem to provide a data communication connection to a corresponding type of communications line. Wireless links may also be implemented. In any such implementation, the communication interface sends and receives electrical, electromagnetic or optical signals that carry digital data streams representing various types of information.

The network link typically provides data communication to the cloud through one or more networks to other data devices. For example, the network link may provide a connection to another computer or remotely located presentation device through a local network (e.g., a LAN) or through equipment operated by a service provider, which provides communication services through a communications network. In preferred embodiments, the local network and the communications network preferably use electrical, electromagnetic, or optical signals that carry digital data streams. The signals through the various networks and the signals on the network link and through the communication interface, which carry the digital data to and from the computer system, are exemplary forms of carrier waves transporting the information. The computer system can transmit and receive data, including program code, through the network(s) and, the network link and the communication interface. Moreover, the network link may provide a connection through a LAN to a user device or client device such as a personal digital assistant (PDA), laptop computer, tablet computer, smartphone, or cellular telephone. The LAN communications network and the other communications networks such as cellular wireless and wifi networks may use electrical, electromagnetic or optical signals that carry digital data streams. The processor system can transmit notifications and receive data, including program code, through the network(s), the network link and the communication interface.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A method of identifying tumor drug resistance for a particular solid tumor of a patient implemented by a clinical MRI scanner, an electronic device comprising a processor, a data input/output device, and a display input/output device, wherein a MRI imaging protocol, a data processing algorithm, a cancer treatment response information diagram, and a method of drug resistance classification are applied to identification of the type of drug resistance during a tumor treatment scheme, and wherein the method comprises the steps of:
a. when the patient is inhaling air, acquiring a first set of tumor multiple image slices and a serial of reference images of the particular solid tumor generated by dynamic contrast enhanced T2-weighted MR imaging technique with a data input/output device;
b. when the patient is inhaling hyperoxic gas with increasing body blood oxyhemoglobin (HbO₂) concentration, acquiring a second set of tumor multiple image slices and a serial of enhanced images of the same particular solid tumor generated by same dynamic contrast enhanced T2-weighted MR imaging parameters with a data input/output device;
c. calculating tumor region of interest (ROI) volume (V) based on intensity threshold of the dynamic contrast enhanced T2-weighted MR imaging data with the processor;
d. computing a tumor volume change ratio (Vt %) based on a reference volume V₀ of the particular solid tumor with the processor;
e. calculating a tumor all voxels' enhanced signal intensity (ΔSI) data with the processor;
f. calculating tumor oxygenated perfusion percentage (OPP %) data based on a single threshold A with the processor;
g. calculating a set of different thresholds of oxygenated perfusion percentage (OPP %) data and recording their location information with the processor;
h. creating special different threshold value pseudo color maps with the processor;
i. plotting OPP % data and Vt % data of the particular solid tumor on the cancer treatment response information diagram with the processor on the display input/output device; and
j. identifying a type of drug resistance of the particular solid tumor based on analyzing the cancer treatment response information diagram with the processor on the display input/output device.

2. The method of claim 1, wherein the oxygenated perfusion percentage data (OPP %) uses a threshold technique in analyzing dynamic contrast enhancement T2 weighted MRI signal for quantitatively measuring high oxyhemoglobin blood distribution of the particular solid tumor for evaluating tumor microcirculation and ability of drug distribution before and during tumor treatment, wherein tumor microcirculation describes a pathophysiological phenomenon of the particular solid tumor, and wherein drug distribution capabilities of the particular solid tumor describes the same pathophysiological phenomenon of particular solid tumor as tumor microcirculation.

3. The method of claim 1, wherein the method further comprises plotting the oxygenated perfusion percentage data OPP % and displaying a reconstruction tumor oxygenated perfusion percentage (OPP %) pseudo color map using multiple threshold value during cancer treatment, wherein the pseudo-color maps of tumor oxygenation perfusion percentage (OPP %) with each measurement point during treatment are used to visualize and assess high OPP % and low OPP % areas of the particular solid tumor, wherein the tumor low OPP % areas are considered the hypoxic areas of the tumor and are targeted with relative high radiation dose for a Biologically-Guided Radiation Therapy.

4. The method of claim 1, wherein the method comprises the construction of a cancer treatment response information diagram, wherein the diagram comprises two independent symmetrical OPP %-Vt % coordinate graphs composing a triangle structure, each OPP %-Vt % coordinate graph recording the different treatment response information, the two OPP %-Vt % coordinate graphs being mirrored and projected to each other.

5. The method of claim 4, wherein the method further comprises integrating tumor volume change information (Vt %) and tumor oxygenated perfusion percentage information (OPP %) into one therapeutic response information point and displaying the point on one of sides OPP %-Vt % coordinate graph for evaluation of treatment response for a cancer therapy scheme.

6. The method of claim 4, wherein the method further comprises plotting the oxygenated perfusion percentage (OPP %) and volume change ratio (Vt %) obtained from the same cancer therapy scheme plotting on the same side of the OPP %-Vt % coordinate graph for evaluation of tumor treatment response information.

7. The method of claim 5, wherein the oxygenated perfusion percentage (OPP %) and volume change ratio (Vt %) obtained during current same cancer therapy scheme for the particular tumor is plotted on the left side of OPP %-Vt % coordinate graph, and wherein the oxygenated perfusion percentage (OPP %) and volume change ratio (Vt %) data obtained from previous treatment records is plotted on the right side of OPP %-Vt % coordinate graph; wherein if the current therapy scheme is a combination of systemic treatment and irradiation treatment, the systemic treatment data is plotted on the left OPP %-Vt % coordinate graph, and the irradiation treatment data is plotted on the right OPP %-Vt % coordinate graph.

8. The method of claim 7, wherein multiple sets of oxygenated perfusion percentage (OPP %) and volume change ratio (Vt %) from multiple measurement points during current cancer therapy scheme are plotted on the one side of OPP %-Vt % coordinate graph for evaluating tumor treatment response information and identifying the type of tumor drug resistance.

9. The method of claim 7, wherein the current cancer treatment scheme is selected from the group consisting essentially of: systemic therapies (chemotherapy, molecular targeted therapy, immunotherapy, gene therapy, photodynamic therapy), local irradiation therapies (radiotherapy, hyperthermia therapy), and systemic therapies-local irradiation therapies combinations.

10. The method of claim 7, wherein at least one previous cancer treatment response record is included the group consisting essentially of: systemic therapies (chemotherapy, molecular targeted therapy, immunotherapy, gene therapy, photodynamic therapy), local irradiation therapies (radiation therapy, hyperthermia therapy), systemic therapies-local irradiation therapies combinations.

11. A method of using tumor oxygenated perfusion percentage (OPP %) and volume change ratio (Vt %) of a particular solid tumor for assisting evidence-based tumor precision medicine, the method comprising:
a. building up a OPP %-Vt % response database of different tumor responses to a treatment scheme, exploring the relationship between the measured tumor response information (oxygenated perfusion percentage (OPP %) and volume change ratio (Vt %) and their clinical outcomes of the treatment scheme;
b. measuring multiple continuous measurements of the oxygenated perfusion percentage (OPP %) and a volume change ratio (Vt %) data with the treatment scheme;
c. determining next treatment plan based on the measurement of individual tumor OPP % and Vt % data and the tumor OPP %-Vt % response database; and
d. wherein the method is performed by one or more electronic devices.

12. The method of claim 11, wherein the oxygenated perfusion percentage (OPP %) and volume change ratio (Vt %) of current response and previous treatment response records are visualized on a cancer treatment response information diagram for reviewing, wherein the diagram comprises two independent symmetrical OPP %-Vt % coordination systems composing a triangle structure, and wherein both OPP %-Vt % coordinate graphs are mirrored and projected to each other.

13. The method of claim 12, wherein the method further comprises plotting the oxygenated perfusion percentage (OPP %) and volume change ratio (Vt %) data obtained during same treatment scheme and plotting on the cancer treatment response information diagram for assisting evidence-based precision cancer treatment.

14. The method of claim 13, wherein the oxygenated perfusion percentage (OPP %) and volume change ratio (Vt %) obtained during one cancer therapy modality for the particular solid tumor is plotted on left OPP %-Vt % coordinate graph, and wherein the data set of oxygenated perfusion percentage (OPP %) and volume change ratio Vt % obtained of another cancer therapy modality for the particular patient is plotted on the right OPP %-Vt % coordinate graph.

15. A method of identifying the type of tumor drug resistance during a treatment scheme for a particular solid tumor, wherein it is applicable to all blood-borne therapies and local irradiation therapy except surgery, the method comprising:
a. completing multiple continuous measurements during the same treatment scheme, wherein each oxygenated perfusion percentage (OPP %) and volume change ratio (Vt %) are obtained and plotted on an OPP %-Vt % coordinate graph of a cancer treatment response information diagram;
b. continuously analyzing the multiple data sets and plotting them on the OPP %-Vt % coordinate graph as the treatment scheme progresses;
c. retrospectively analyzing at least the latest two consecutive detection points of tumor OPP %-Vt % data, wherein if the consecutive measurements show that the tumor increases (Vt % is positive value) or the tumor continues to shrink (Vt % is negative value) and their difference is less than the critical value (3%), it is determined that the tumor is developing treatment resistance, and wherein identifying the type of tumor treatment resistance comprises the following steps:
i. if at least two consecutive measurements show that the tumor increases (Vt % is positive value) or the tumor continues to shrink (Vt % is negative value) and their difference is less than the critical value (3%) and the oxygenation perfusion percentage (OPP %) are all less than a critical value (5%), the resistance can be identified as the type of a pharmacological/physiological factors (low drug distribution and poor tumor microcirculation); and
ii. if at least two consecutive measurements show that the tumor increases (Vt % is positive value) or the tumor continues to shrink (Vt % is negative value) and their difference is less than the critical value (3%) and the oxygenation perfusion percentage (OPP %) data are all greater than a critical value (20%), the drug resistance can be identified as the type of a cell-specific factors; and
d. wherein during tumor treatment, drug resistance of the solid tumor and its type may change dynamically in response to different treatment schemes, and multiple measurements are necessary for timely and accurate identification of tumor drug resistance and its type.

16. The method of claim 15, wherein the identification of drug resistance and its type is applicable to all “blood-borne therapies” modalities and “local irradiation therapy” modalities except surgical treatment. Measurement of the drug resistance during the treatment period can be arranged by the clinician according to the tumor response.

17. The method of claim 15, wherein the method of identification of drug resistance and its type is applicable to monitoring tumor normalization vasculature treatment via anti-angiogenic therapy, and wherein the parameter of oxygenated perfusion percentage (OPP %) is used to evaluate tumor vasculature remolding.

18. The method of claim 15, wherein the standard of critical values for classifying the type of drug resistance is determined by the results of clinical statistics data, and the threshold values for classification is modified based on clinical data and are related to the tumor site and pathological stage.
